(19) United States
(12) Patent Application Publication
(10) Pub. No.: US 2026/0014180 A1
(43) Pub. Date: Jan. 15, 2026

(54) BIOACTIVES AND THEIR USES

(71) Applicant: AREPA IP LIMITED, Auckland (NZ)

(72) Inventors: Angus Ross BROWN, Auckland (NZ); Stephen John BLOOR, Auckland (NZ)

(21) Appl. No.: 18/842,335

(22) PCT Filed: Feb. 28, 2023
(86) PCT No.: PCT/NZ2023/050027

§ 371 Date: Aug. 28, 2024

(30) Foreign Application Priority Data
Feb. 28, 2022 (NZ) 785645

(51) Int. Cl. A61K 31/7032; A61K 36/185; A61P 25/00; A61P 25/22

(52) U.S. Cl. CPC A61K 31/7032; A61K 36/185; A61P 25/00; A61P 25/22

(57) ABSTRACT

This invention relates broadly towards sarmentosin and its esters and their new use as MAO inhibitors and use as treatment or prevention of various associated conditions related to MAO activity.

### CROSS-REFERENCE TO RELATED APPLICATIONS

This application is the U.S. national phase entry of PCT/NZ2023/050027 filed Feb. 28, 2023, which claims the benefit of NZ Application No. 785645 filed Feb. 28, 2022.

### TECHNICAL FIELD

This invention relates broadly towards sarmentosin and its esters and their new use as MAO inhibitors and use as treatment or prevention of various associated conditions related to MAO activity.

### BACKGROUND ART

Monoamine oxidase (MAO) is an enzyme which catalyses the oxidative deamination of amines such as dopamine and serotonin. The enzyme appears as two isozymes, MAO-A and MAO-B. Monoamine inhibition can be reversible or irreversible and can either act non-selectively, affecting both isoforms, or selectively, affecting only one isoform. They play a major role in the metabolism of both dietary and endogenous monoamines (Yamada & Yasahura, 2004).

The MAO enzymes are of interest as targets for nutraceuticals, functional foods and drug discovery. This is because, for example, MAO-A is involved in psychiatric conditions and depression and MAO-B is linked to neurological disorders such as Parkinson's and Alzheimer's diseases.

In 1994, Delumeau et al. summarised, from various human and animal studies, that MAO inhibitors appear to improve cognitive function as well as helping prevent cognitive disorders like Alzheimer's disease, and therefore such inhibitors once identified, present real therapeutic benefit for their cognitive enhancing properties.

A further review by Zhiyou Cai in 2014 highlights various MAO inhibitors which could present therapeutic agents for Alzheimer's disease.

A further review by Carradoni et al, 2018 highlights a range of MAO inhibitors in the patent literature from 2015-2017, including a number of newly synthesised and naturally occurring chemicals from plant materials.

By way of example, Phenelzine is an irreversible, non-selective MAO inhibitor, which inhibits both MAO-A and B for up to three weeks.

In contrast, toloxatone, a reversible inhibitor of MAO-A, inhibits MAO-A for only six hours before activity returns to baseline values.

Selective reversible inhibitors are preferable as they avoid toxic build-up of dietary monoamines, in the digestive tract and if this continues for prolonged periods tyramine can accumulate to dangerous levels, potentiating a hypertensive crisis. It is therefore important to identify reversible and/or selective MAO inhibitors.

Isocarboxazid, phenelzine and tranylcypromine appear to have fairly comparable properties (Mallinger & Smith 1991). They are readily absorbed and reach peak concentrations in 1-2 hours. Elimination is also swift, with half-lives in the range of 1.5 to 4 hours (especially short for tranylcypromine).

Moclobemide is a more recent MAO inhibitor drug and is readily absorbed and reaches peak plasma concentrations in approximately 1 hour. Metabolism is rapid and complete with an elimination half-life in the range of 1-3 hours.

Deprenyl (also known as selegiline) is a MAO-B inhibitor drug which has efficacy for treating depression and Parkinson's disease at dosages between 1.25 mg to 10 mg per day. However, there can be significant side effects including suicidal thoughts, and hypertension.

In fact, many MAO inhibitor drugs have fallen out of favour for treatment of depression due to side effects from adverse drug and food interactions. Their primary use remains in the treatment of Parkinson's Disease.

As a further example, U.S. Pat. No. 8,367,121B2 describes a nutraceutical-based approach to attenuating the process leading to Parkinson's Disease, relying in part through selection of known MAO inhibitors (see para 0045-0047).

Because of MAO's role in modulating dopamine and serotonin, the Mayo Clinic website (link) also highlights that MAO inhibitors have been useful tools to improve mental health conditions, mood or address conditions like depression or anxiety. Various MAO inhibitors have been approved by the FDA including isocarboxazid (Marplan), Phenelzine (Nardil), Selegiline (Emsam), and Tranylcypromine (Parnate).

Studies on diverse sources such as cigarette smoke and coffee have found a class of amines known as beta-carboline alkaloids to be inhibitors of MAO (Herraiz, 2007).

A Madrid-based research team has shown that a number of fruits and fruit juices/extracts contain beta-carbolines (betaCs) (Herraiz, 2006, 2018). When they compared a range of betaCs they found potent MAO activity in some of the betaCs.

There is some supportive evidence of the benefits of anthocyanin-rich blackcurrants on mood and cognition. Two studies have been published showing the effects of acute blackcurrant (Ribes nigrum) supplementation on healthy humans.

Blackcurrant juice is rich in anthocyanins and other flavonoids. Although these phenolics were shown to have some MAO inhibitory activity, it is possible that there are also other unidentified components which may confer additional, potentially more potent MAO inhibitory activity, and therefore may be of commercial and therapeutic importance. Watson et al 2015 highlighted that there was no significant MAO-B inhibition following consumption of an anthocyanin rich blackcurrant extract, suggesting removal or degradation of an unknown bioactive may be responsible for much of the MAO activity. Indeed, it has been thought for some time that some betaCs in blackcurrant could be a strong candidate for an observed MAO inhibitory activity sometimes observed in BC juice (Budzikiewicz, 1994, and Herraiz, 2006, 2018).

In particular, it is of real interest to identify compounds with MAO inhibitory activity in fruit that are naturally present that could then be utilised, isolated, protected, extracted, synthetically replicated or recombinantly produced/expressed through cellular fermentation, or heightening the levels in the fruit or fruit juice of the compound(s) via selective breeding or other known genetic modification techniques.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Further aspects and advantages of the present invention will become apparent from the ensuing description which is given by way of example only.

### DISCLOSURE OF THE INVENTION

According to a first aspect of the present invention there is provided a use of sarmentosin or an ester(s) thereof for inhibiting monoamine oxidase enzyme A (MAO-A) and/or monoamine oxidase enzyme B (MAO-B).

According to a first aspect of the present invention there is provided a use of sarmentosin or an ester(s) thereof for preventing or treating a disease or condition, or for the maintenance or improvement of a non-clinical cognitive state or condition associated with MAO-A or MAO-B enzyme activity.

According to a further aspect of the present invention there is provided a composition formulated as a food or beverage, pharmaceutical composition, nutraceutical or an extract from a plant material, wherein the composition includes sarmentosin or an ester(s) thereof, and when the composition is used for inhibiting monoamine oxidase enzyme A (MAO-A) and/or monoamine oxidase enzyme B (MAO-B).

According to a further aspect of the present invention there is provided a use of sarmentosin or an ester(s) thereof, or a plant material, an extract or a composition containing same, in the manufacture of a medicament for the inhibition of monoamine oxidase enzyme A (MAO-A) and/or monoamine oxidase enzyme B (MAO-B) in a person in need thereof.

According to a further aspect of the present invention there is provided a method of inhibiting monoamine oxidase enzyme A (MAO-A) and/or monoamine oxidase enzyme B (MAO-B) by administering to a person in need thereof with an effective amount of sarmentosin or ester(s) thereof, or a plant material, an extract or a composition containing same.

According to a further aspect of the present invention there is provided a blackcurrant fruit, blackcurrant juice, extract, or isolate including an effective amount of sarmentosin or ester(s) thereof, when used for inhibiting MAO-A or MAO-B.

As will be further discussed and exemplified below a key aspect of the present invention is the newly identified use of sarmentosin and its ester(s) as a MAO-A and/or MAO-B inhibitor. To the best of the Applicant's knowledge, this important and unexpected activity of sarmentosin or its ester(s) was not previously known. Surprisingly, the MAO inhibitory activity in blackcurrant was shown not to be associated with the anthocyanins/polyphenols and not β-carbolines, as was expected. This opens up huge potential for sarmentosin and its ester(s) as bioactives to be used in their purified form, or as part of a product using a natural source such as blackcurrants ensuring retention of said bioactives, as an new way to inhibit MAO-A and/or MAO-B, and the many clinical and non-clinical diseases and conditions associated with these enzymes. Another key advantage of these bioactives is that they exist naturally and show no adverse effects like other commercially developed MAO inhibitors. Sarmentosin and its esters also appear to be reversible MAO Inhibitors. The inventors are the first to discover significant MAO activity in blackcurrants specifically arising from these bioactives, and therefore to harness the specific use within blackcurrant or other sources for MAO inhibition, or to enhance, concentrate, fortify, or protect the sarmentosin for an enhanced use, potency or effects. It also opens up new possibilities to purify sarmentosin in an extract or to isolate as a fully purified bioactive for new uses, and the opportunity to synthetically manufacture the bioactive for beneficial uses. The invention will be further discussed in the Preferred Embodiments and Detailed Description below.

### Definitions

Throughout this specification the term “sarmentosin” should be taken as meaning a gamma hydroxy nitrile glycoside (chemical name: 4-(beta-D-glucopyranosyloxy)-2-(hydroxymethyl-2-butenenitrile)), or derivatives thereof as shown in the general chemical structure below:

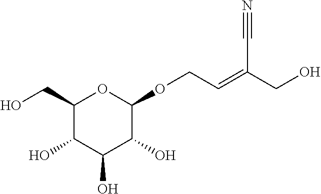 [SMILES: N#C/C(=C\CO[C@@H]1O[C@H](CO)[C@@H](O)[C@H](O)[C@H]1O)CO]

Throughout the present invention the term “sarmentosin ester” or “ester thereof” should be taken as meaning nigrumin-p-coumarate, nigrumin caffeate and/or nigrumin ferulate or derivatives thereof as shown in the general structures below. These are phenolic acid derivatives of sarmentosin.

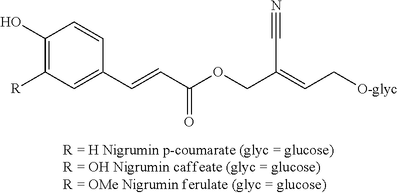

Throughout this specification the term “extract” should be taken as meaning a preparation containing the sarmentosin or an ester thereof taken, isolated, removed, purified or otherwise extracted from a selected source in a more concentrated form or more purified form compared to how it is naturally found. For example, the extract may be in liquid form such as a juice, a juice concentrate, or a dried format such as a powder, tablet or capsule. The extract may contain other bioactives or components from the selected source, or may be combined with other extracts, ingredients or products as desirable. Alternatively, the extract containing the sarmentosin may be in a fully purified or semi purified form to the extent that it becomes a purified isolate, and still be considered an extract for the purposes of this invention.

Throughout this specification the term “composition” should be taken as meaning a combination of the sarmentosin or an ester thereof, in an admixture together with other ingredients or constituents. The composition, for example, may be in the form of a food or beverage, pharmaceutical formulation, nutraceutical, supplement, or natural extract and so forth without departing from the scope of the invention.

Throughout this specification the term “plant material” should be taken as meaning any biological vegetation including roots, leaves, seeds, seedlings, fruit, stalks and so forth.

Throughout this specification the term “effective amount” should be taken as meaning the amount of a compound that, when administered to a human or other mammal for improving, treating, preventing or delaying a state, disorder or condition, is sufficient to affect such treatment. Of course, the “effective amount” can vary based on the compound, the particular condition and its severity as well as the age, physical state, or weight of the mammal to be treated. Preferred effective amounts, including dosages, are discussed further below.

Throughout this specification the term “monoamine oxidase” or “MAO” enzyme should be taken as meaning an enzyme which catalyses the oxidative deamination of amines such as dopamine and serotonin. The enzyme appears as two isozymes, MAO-A and MAO-B.

Throughout this specification the term “MAO inhibitor” should be taken as meaning a type a chemical, drug, substance, extract or bioactive that has partial or full inhibitory activity to one or both monoamine oxidase enzymes, namely monoamine oxidase A (MAO-A) and monoamine oxidase B (MAO-B). This inhibition may be reversible or irreversible.

Throughout this specification the term “nootropic” should be taken as meaning a substance that improves or supports cognition, memory and/or facilitates learning.

### PREFERRED EMBODIMENTS

### MAO Inhibition Activity

Preferably the sarmentosin, its esters thereof or extract containing same provides reversible MAO inhibition.

The profile of MAO-B inhibition of blackcurrant juice bears a notable similarity, to pharmaceutical reversible MAO-B specific inhibitors such as lazabemide, which have shown a rapid inhibition of MAO-B in platelets of >90% at 30 min post dose, with maximal inhibition subsiding 16 h post dose and full restoration of enzyme activity returning 48 h post dose following a 100 mg dose of lazabemide (Dingemanse et al., 1997).

Advantageously, whilst expressing similar MAO inhibitory effects as pharmaceutical drugs, blackcurrant supplementation has shown no shown detrimental side effects from its MAO inhibition (Braakhuis et al., 2020), and appears to advantageously offer reversible inhibition of MAO enzymes. Furthermore, blackcurrant fruit, extracts from blackcurrant, or the sarmentosin extracted or isolated from blackcurrant also appears to be particularly advantageous because the strong safety history of blackcurrant as a food, further suggesting sarmentosin itself will have a safe profile as a MAO inhibitor. In contrast, very few MAO inhibitor drugs are still used due to safety profiles. To the best knowledge of the inventors, laxabemide was never marketed.

Preferably, the sarmentosin or ester(s) thereof have both MAO-A and MAO-B inhibition activity. This is supported and discussed in Example 2.

Having activity to both MAO enzymes may be very beneficial as they have strong affinities for different substrates. MAO-A shows greater affinity for hydroxylated amines such as noradrenaline and serotonin, whereas MAO-B shows greater affinity for non-hydroxylated amines such as benzylamine and beta-phenylethylamine (PEA). Additionally, dopamine and tyramine show similar affinity for each enzyme form, meaning inhibition of both may be beneficially required to modulate their availability.

### Composition and Formats

The composition may be in a variety of formats without departing from the scope of the invention.

Preferably, the sarmentosin or its ester(s) thereof is within a plant material, an extract or a composition.

A particularly preferred format is a beverage or drink, or powder supplement; however, a number of other options may be achieved easily by a skilled person.

Preferably, the sarmentosin or its esters are provided in a food or beverage.

More preferably, the food or beverage is based on blackcurrant fruit, blackcurrant juice, blackcurrant extracts (liquid or powder format), and so forth. Similarly, one would appreciate that any other fruit source containing sarmentosin or its ester(s) would be applicable. Alternatively, one may choose to extract, isolate or synthetically make sarmentosin, and then add it to any such juice or composition type and not depart from the scope of the invention.

As the inventors have identified the presence and MAO inhibitory activity of sarmentosin and its esters in blackcurrant fruit (and its extracts), a strong commercial preference is to utilise the existing bioactivity for this new use in blackcurrant based or blackcurrant containing products. It should be appreciated that either the existing levels of sarmentosin or its ester(s) may be used, or further fortified, concentrated, boosted with additional amounts of sarmentosin.

Alternatively, the sarmentosin or its ester(s) are provided in a nutraceutical or therapeutic format such as a powder, tablet or capsule. Again, these formats may be derived from or contain blackcurrant material.

As previously highlighted, in another embodiment the whole fruit or part thereof or whole plant material or part thereof (e.g. blackcurrant) containing said sarmentosin or its ester(s) is used to achieve the new uses or methods described herein.

Alternatively, the sarmentosin or its ester(s) is provided as an extract from a plant source.

Preferably, the extract is a polar fraction or component extracted from the selected source of the sarmentosin.

Preferably, the extract is non-polyphenolic.

Preferably, the extract containing the MAO inhibition activity from sarmentosin or its ester(s) also includes BetaCs.

Preferably, the extract is a juice or a concentrated version thereof.

More preferably, the juice extract is from blackcurrant fruit.

In a further embodiment, someone skilled in the art could appreciate the invention may be leveraged through the development of a new plant variety with sarmentosin or one of its esters with levels greater than normally or naturally present. For example, the plant variety may that of a blackcurrant plant (Ribes nigrum) which already has naturally occurring, albeit at relatively minimal levels, of sarmentosin and its esters which is then selectively bred to have increased levels of the bioactive(s) and then utilised for the applications described herein.

Preferred concentration of sarmentosin or ester(s) in the extract or composition

Preferably, the extract or composition has at least about 0.005 mg, or more preferably 0.05 mg/g sarmentosin per gram (or mg/g) of the extract or composition.

This is indicative of a minimum level of sarmentosin in various blackcurrant juices tested by the inventors-see Example 4, although noting there are differences in the bioactive levels from different juices and other samples tested. Through experience, the inventors have discovered that various factors such as high temperature processing (e.g. during pasteurization), product time on shelf and storage conditions, fruit seasonality, and plant breeds can all have an impact on the level of sarmentosin and its ester(s), and in some cases may even be negligible or non-existent.

Therefore, there may be a need or advantage to concentrate, fortify, add or supplement sarmentosin in a composition or extract, or to select for plants or fruit that advantageously have higher levels of sarmentosin. Similarly, there may be an advantage to stabilise, protect, preserve, restore or further increase sarmentosin levels beyond what is present, or was present, naturally in blackcurrants or other sources thereof. Similarly, sarmentosin may ultimately be added to anything for the intended uses as described herein without departing from the scope of the invention.

In preferred embodiment, the extract or composition has at least about 0.005, 0.01, 0.02, 0.05, 0.1, 0.15, 0.2, 0.25, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, or 17.0, 18.0, 19.0, 20.0, 21.0, 22.0, 23.0, 24.0, 25.0, 30.0, 40.0, 50.0, 60.0, 70.0, 80.0, 90.0, 100.0 or 200.0 mg sarmentosin thereof per gram of extract or composition.

More preferably, the extract or composition has at between about 0.005-70.0 mg sarmentosin per gram (or mg/g) of the extract or composition.

As discussed further in the Preferred Dosages section, a preferred minimum dosage may be about 20 mg per day of sarmentosin. If a blackcurrant juice had about 0.3 mg/ml sarmentosin, a serving size of 86 ml of that juice would achieve this minimum dosage. A person skilled in the art would appreciate the concentration of sarmentosin may be suitably adjusted in a desired extract or composition using conventional methods and practices to achieve effective amounts/dosages and other criteria.

Similar embodiments may also be envisioned for the sarmentosin esters shown for example in Example 4. Whilst the levels of the esters are lower comparatively to sarmentosin, someone skilled in the art would appreciate that there are means to increase the levels of these in an extract or composition if they so desired.

### Sources of Sarmentosin or its Esters

Preferably, sarmentosin may be extracted or isolated from a number of sources without limitation. In the past, sarmentosin esters have been identified in blackcurrant seeds ((Lu et al., 2002), and sarmentosin has been identified in Kalanchoe species (Fernandes et al, 2021).

Here, the inventors have now surprising discovered sarmentosin and its esters in blackcurrant fruit, and fruit extracts (such as dried extracts or juices). Therefore, preferably sarmentosin is extracted or isolated from blackcurrant fruit. The extract may also include other components from the source (such as blackcurrant) as this may provide additional benefits from bioactives or micronutrients. The inventors foresee that in an alternative use embodiment, a whole fruit or plant material may be used to provide the beneficial MAO inhibitory effect from sarmentosin or its esters, so long as the material indeed has this beneficial bioactive which is the subject of this patent application.

CN102659860 and CN101974044B both describe other methodologies for extracting sarmentosin from other plant sources. These present alternative approaches to extract or isolate sarmentosin for the new uses of the present invention. Whilst these may be suitable sources, it should be appreciated that other plants may also contain similar or higher levels of sarmentosin or its esters which here have been shown to have MAO inhibition activity.

Alternatively, the source of sarmentosin or its esters may also be synthetically produced through known methodologies, or via microbial fermentation production either from microbes that naturally express sarmentosin, or via genetic modification using constructs that are coded to recombinantly express sarmentosin in standard cellular culture methodologies.

A person skilled in the art would appreciate that there are other means to achieve commercial production of sarmentosin beyond what is naturally found currently. This may be through development of a suitable plant variety, either through genetic engineering, via cross-breeding or multi-generational selection procedures to produce new varieties with high levels of sarmentosin or its esters. In other words, the present invention may be achieved through artificially producing sarmentosin or its esters at high purity, and provided, stored, or sold as a purified form or suitably provided within a pharmaceutical composition. This may be particularly useful for the pharmaceutical industry where stringent manufacturing requirements are required.

In the embodiment of a pharmaceutical drug, suitable formats including but not limited to transdermal creams or patches, parenteral or subcutaneous injection, pills, capsules and so forth are all possible routes of administration.

### Potential Options for Methods of Manufacture

The juice from the Ribes nigrum may be manufactured according to standard practice. For example, a juice concentrate may be obtained by evaporation under vacuum of the juice to yield a concentrate with approximately 10x the concentration of sarmentosin.

A powdered form of the juice may be produced by freeze-drying or spray drying the juice. Therefore, one skilled in the art would appreciate that the invention may be provided in various different dried formats, such as capsules, tablets, powders, and so forth.

Pure sarmentosin may be obtained by chromatographic separation of the juice or juice concentrate using reversed phase chromatography with multiple steps until pure sarmentosin is obtained.

Those skilled in the art would appreciate there are also standard methodologies available to chemically synthesise compounds such as sarmentosin or its esters. Such processes are encompassed within the present invention.

Likewise, cellular expression systems are nowadays used routinely to produce desired compounds at scale. A natural or modified bacterial system, for instance, could be selected or produced that overexpressed large amounts of sarmentosin, and then sarmentosin could be purified from the cell culture.

Further examples of how compositions containing sarmentosin may be prepared are in the Best Modes section of the specification.

### Methods of Use and Treatments

MAO inhibitors have already shown promise and are associated with a range of diseases and conditions as highlighted throughout this specification. As such, there is a strong expectation that sarmentosin or one of it(s) identified esters, now found to have MAO inhibition activity, are very likely to have new application to treat or prevent these same or similar conditions, both clinically and non-clinically. It should be appreciated that all MAO inhibitors generally share the same mechanism of action and are comparable when it comes to efficacy.

Preferably, the use or methods of treatment as described herein modulates, maintains or increases a neurotransmitter selected from the group consisting of dopamine, serotonin, adrenalin (noradrenalin or norepinephrine) and tyramine.

### Potential Clinical Uses

Preferably, the use or methods of treatment as described herein are for the treatment or prevention of a disease or condition is any neurological or psychiatric condition or ailment associated with MAO-A or MAO-B enzyme activity in a person in need thereof.

Preferably, the sarmentosin or its ester(s) are used for treating or preventing depression, atypical depression, panic, social anxiety, bipolar disorder (especially the depressive phase), post-traumatic stress disorder (PTSD), Obsessive Compulsive Disorder (OCD), adult ADHD, Alzheimer's disease, Dementia, Parkinson's disease and/or Huntington's disease.

### Non-Clinical Uses

Additionally, MAO inhibitors have shown potential application to a range of non-clinical uses, as discussed throughout this specification. For example, Rhodiola, also known as golden root Rhodiala, which contains MAO inhibitors, has been used to help with mood, social anxiety, reduce fatigue and improve exercise performance.

Preferably, the sarmentosin or its ester(s), are used for improving, restoring or supporting non clinical effects such as mood, anxiety, social anxiety, fatigue, cognitive performance, exercise performance, attention/alertness, calmness, mental clarity, executive function, working memory, secondary memory, mood, stress and/or stress reactivity or to provide a nootropic effect.

A skilled person in the art would appreciate other non-clinical uses related to cognitive and mental support would also fall within the scope of the invention and its uses.

The sarmentosin may also be mixed with other nootropics or bioactives that are known to be beneficial for neurological state or prevention of conditions or disease.

### Preferred Dosage

The inventors have conducted preliminary tests (see Example 5) to assess the likely efficacious dosage of sarmentosin or its ester(s) for clinical uses, and potentially also a guide for non-clinical uses too. This was achieved through comparative enzyme inhibition analysis to a known commercial MAO-B inhibitor (Deprenyl) used for clinical purposes. Extrapolating the data from Deprenyl in Example 5 and dosage guidance (http://www.drugs.com/dosage/selegiline) suggests an equivalent initial daily dosage of 27.5 mg sarmentosin for clinical uses and then after six weeks the daily dosage may be increased to 65 mg if needed.

Therefore, for a clinical usage similar to Deprenyl, the daily dosage may preferably be at least 20 mg sarmentosin or its ester(s).

More preferably, the daily dosage is between 20-200 mg sarmentosin or its ester(s).

More preferably, the daily dosage is about 20, 25, 30, 35, 40, 45, 50, 55, 60, 65, 70, 75, 80, 85, 90, 95, 100, 105,110, 115, 120, 125, 130, 135, 140, 145, 150, 155, 160, 165, 170, 175, 180, 185, 190, 195, or 200 mg sarmentosin or its ester(s).

More preferably, the daily dosage is between 20 to 200 mg sarmentosin or its ester(s).

Another preferred daily dosage may be in the order of at least 110 mg/60 kg body weight.

In a more preferred embodiment, the daily dosage of sarmentosin or its esters thereof is between 110-130 mg/60 kg body weight.

In a non-clinical setting, preferred dosages may be substantially less than clinical settings.

Preferably for non-clinical uses (for example for preventative, supportive or maintenance effects towards mood or cognition—as discussed above), a serving size/daily serving may be as low as 1 mg-20 mg, although higher dosages similar to the preferred clinical dosages may also be useful and within the scope of the invention.

### Key Advantages of the Present Invention

The present invention may include one or more of the following advantages, without limitation:
Identification of a new use of sarmentosin and its esters as a MAO inhibitor with potentially significant opportunity for therapeutic and non-clinical uses.
Various embodiments of the present invention may beneficially leverage a naturally occurring source of sarmentosin or its esters, owing to the fact it is present in several plant materials including blackcurrants.
Preliminary evidence suggests the sarmentosin has minimal or no disadvantageous side effects.
Preliminary evidence suggests that sarmentosin advantageously has both inhibitory activity towards both MAO-A and MAO-B, opening up additional uses.
Preliminary evidence suggests that sarmentosin advantageously reversibly inhibits MAO-A and MAO-B.

### BRIEF DESCRIPTION OF THE FIGURES

Further aspects of the present invention will become apparent from the ensuing description which is given by way of example only and with reference to the accompanying Figures.

FIG. 1 Referring to Example 1, BetaCs reported from blackcurrant juice. Compound 1 (LHS) and compound 2 (RHS).

FIG. 2 Referring to Example 1, a third betaC found in blackcurrant (Compound 3).

FIG. 3 Referring to Example 1, LCMS analysis of test blackcurrant juice.

FIG. 4 Referring to Example 1, Chromatograms of Neuroberry™ juice samples, 1, 2 and 3 (top to bottom).

FIG. 5 Referring to Example 3, levels of 4HQ seen in fractions (lower value indicates more inhibition)

FIG. 6 Referring to Example 3, prep HPLC trace (UV). Fractions 18-23 are collected from 7-11 minutes.

FIG. 7 Referring to Example 3, levels of 4HQ seen in fractions from large scale prep hplc (lower value indicates more inhibition)

FIG. 8 Referring to Example 3, showing the chemical structure of sarmentosin.

FIG. 9 Referring to Example 3, showing the chemical structures of nigrumin compounds.

FIG. 10 Referring to Example 3, activity vs sarmentosin content for fractions 17-22 from prep HPLC.

FIG. 11 Referring to Example 3, H NMR spectrum of prep HPLC fractions.

FIG. 12 Referring to Example 3, H NMR spectra, detail of 5-10 ppm region.

FIG. 13 Referring to Example 3, levels of 4HQ in bioassay of reversed phase fractions rp-1 through 11.

FIG. 14 Referring to Example 4, RP10 fractions 4HQ (S9 Bioactivity for preparative HPLC fraction from RP10)

FIG. 15 Referring to Example 4, Caumaroyl and feruloyl sarmentosin content in RP10 HPLC fractions (fractions 12-15 have higher peak areas than shown)

FIG. 16 Referring to Example 4, Caffeoyl sarmentosin peak area content in RP10 HPLC fractions

FIG. 17 Referring to Example 5 A) Dilution series for sarmentosin. 50% inhibition occurs at about 400 ng/ml. B) Dilution series for Deprenyl. 50% inhibition occurs at about 10-15 ng/ml.

FIG. 18 Referring to Example 6, Plot of sarmentosin(S) concentration in the sample vs. the concentration of each sample needed for 70% inhibition of MAO-b.

FIG. 19 Referring to Example 7, Flow diagram of progress through the phases of the randomised cross-over trial with two format groups.

FIG. 20 Referring to Example 7, Procedures carried out on Trial Day 1 and 2 of the intervention study.

FIG. 21 Referring to Example 7, Concentrations (means±SEM) of total anthocyanins (A), cyanidin derivatives (B, C, D) and delphinidin derivatives (E, F) in plasma before (0 min) and after consuming blackcurrant beverage interventions. Data are presented as cyanidin 3-O-glucoside equivalents.

FIG. 22 Referring to Example 7, Sarmentosin concentrations (mean±SEM) in plasma before (0 min) and after consuming a single dose of blackcurrant juice or powder.

FIG. 23 Referring to Example 7, Platelet monoamine oxidase (MAO)-B activity and blood glucose concentrations before (0 min) and after consuming blackcurrant juice (A, C) or powder (B, D) and their corresponding placebos. Values are mean±SEM. * Indicates significant difference from 0 min (p<0.05).\hat{ } Indicates significant difference from placebo intervention at the same time point.

FIG. 24 Referring to Example 7, Visual Analogue scores for Interested/Bored (A, B), Friendly/Unfriendly (C, D), Happy/Sad (E, F) and Calm/Excited (G, H) items from the Bond-Lader Questionnaire given by participants before (0 min) and after consuming blackcurrant juice or powder and their corresponding placebos. Values are mean±SE. * Indicates significant difference placebo intervention at the same time point.

FIG. 25 Referring to Example 7, Spearman's rank-sum correlation coefficients for neurotransmitters showing a significant correlation (*p<0.05, **P<0.01) with MAO-B inhibition after consumption of either a blackcurrant juice intervention vs a placebo, or a blackcurrant powder intervention vs a placebo.

FIG. 26 Referring to Example 7, Concentration difference (A ng/ml) (means±SEM) of circulating plasma neurotransmitters between timepoint (t)=0 and t=10, 20, 120, 240 and 480 mins after consumption of either a blackcurrant juice intervention vs a placebo, or a blackcurrant powder intervention vs a placebo. (A) 3,4-dihydroxyphenylacetic acid (DOPAC) juice intervention, (B) DOPAC powder intervention, (C) homovanillic acid (HVA) juice intervention, (D) HVA powder intervention, (E) vanillylmandelic acid (VMA) juice intervention, (F) VMA powder intervention, (G) dopamine (DA) juice intervention and (H) DA powder intervention.

FIG. 27 Referring to Example 7, Concentration difference (A ng/ml) (means±SEM) of circulating plasma neurotransmitters between timepoint (t)=0 and t=10, 20, 120, 240 and 480 mins after consumption of either a blackcurrant juice intervention vs a placebo, or a blackcurrant powder intervention vs a placebo. (A) 5-hydroxyindoleacetic acid (5-HIAA) juice intervention, (B) and 5-HIAA powder intervention. * indicates significant difference between the intervention and the placebo at the same time point (p<0.05).

FIG. 28 Referring to Example 7, Concentration difference (Δ ng/ml) (means±SEM) of circulating plasma neurotransmitters between timepoint (t)=0 and t=10, 20, 120, 240 and 480 mins after consumption of a blackcurrant powder intervention vs a placebo. (A) xanthurenic acid (XA), (B) metanephrine (MN), (C) normetanephrine (NM) and (D) kynurenic acid (KA).

FIG. 29 Referring to Example 7, Concentration difference (Δ ng/ml) (means±SEM) of circulating plasma neurotransmitters between timepoint (t)=0 and t=10, 20, 120, 240 and 480 mins after consumption of either a blackcurrant juice intervention vs a placebo, or a blackcurrant powder intervention vs a placebo. (A) 3-methoxy-4-hydroxyphenylglycol (MHPG) juice intervention, (B) MHPG powder intervention, (C) 3,4-dihydroxyphenylglycol (DHPG) juice intervention, (D) DHPG powder intervention, (E) phenylalanine (PHE) juice intervention and (F) PHE powder intervention. * indicates significant difference between the intervention and the placebo at the same time point (p<0.05).

FIG. 30 Referring to Example 7, Correlation circle plots of standardised neurotransmitter concentrations as dependent variables for blackcurrant (A) and placebo (B) beverages (juice and powder combined). Only the 12 variables with the strongest contributions to the definition of the principal components are displayed. Variables with warm and cool colours higher and lower weights, respectively. The coordinates of the variables correspond to their correlation coefficient values and the angle between two variables defines their relationship: If the angle between two variables is sharp the correlation is positive, if the angle is obtuse the correlation is negative and if the angle is right the correlation is null.

### DETAILED DESCRIPTION

### Example 1—Review of Previous R&D and Preliminary Investigations of Beta-Carbolines

### Overview

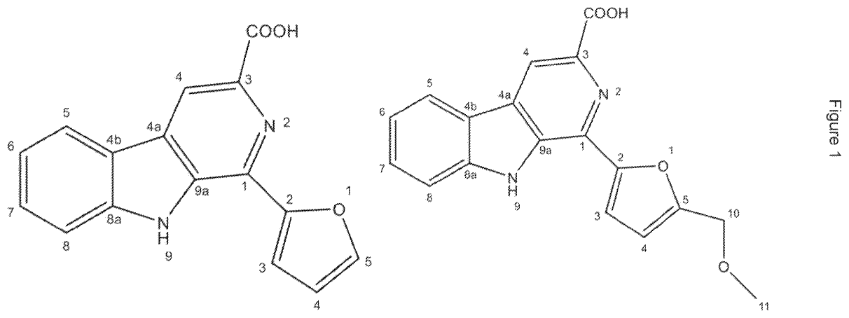

Previous studies have indicated that consumption of blackcurrant (BC) juice affects the monoamine axis in human subjects. In 1994, Budzikiewicz showed that BC contains two beta-carbolines (betaCs)—see FIG. 1. This class of compounds are known to interact with MAOs. As noted before, in 2006 and 2018 Herraiz et al. showed that a variety of juices and fruits, including BC contain beta carbolines (betaCs). When they compared a range of betaCs they found potent MAO activity in some of the betaCs. MAO-B activity was shown to be inhibited by consumption of the BC juice. The activity was suggested to be reversible and non-selective but with possible greater MAO-B affinity. These betaC compounds are present at very low levels in the juice, yet the inventors considered betaCs as the obvious candidates for the MAO inhibition activity in the BC juice.

Example 1 includes:
developing analytical methods to detect low levels of beta-carbolines in BC samples. This would involve processing of a bulk BC juice or frozen berries (10 kg) to specifically extract the betaCs.
preparing samples with enhanced betaCs using buffered reactions of common precursors
use of LC-MS and LC-HRMS to detect betaCs in juice and enhanced samples
   Method and Results of Analysis of betaCs in BC.

Analysis of these betaCs is most conveniently performed using LCMS. Alkaloids tend to ionise well in mass spectrometry and specific MRM analysis allows more precise analysis in complex mixtures. Initially samples of juice were extracted with diethyl ether, this yielded ether extracts enriched in the betaCs. These were then subjected to LCMS and the peaks corresponding to the masses of the two known betaCs identified (279 and 323, MH+ for 1 and 2).

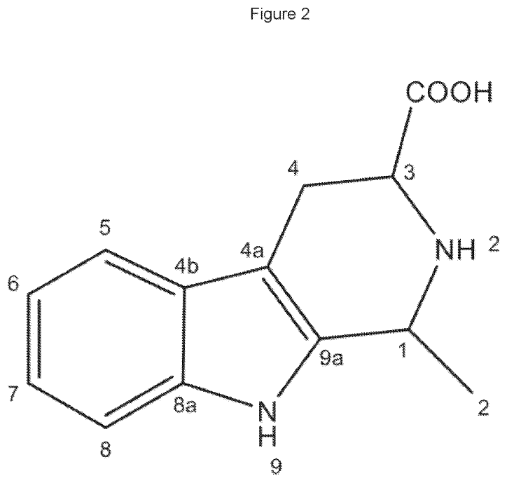

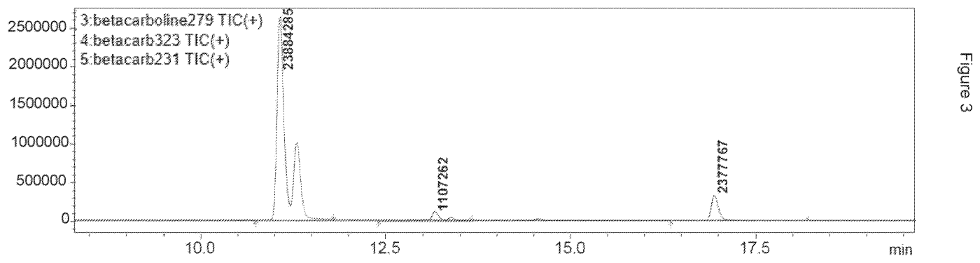

During this work we also noted the presence of another betaC with a MH+ of 231. identified as methyl tetrahydro-beta-carboline (see FIG. 2).

Compounds 1-3 (in FIGS. 1-2) can be derived from tryptophan. The lab reaction of tryptophan with an aldehyde to yield this type of compound is known as the Pictet-Spengler reaction. Even under quite mild conditions like those in a juice, tryptophan will react with aldehydes to give these products.

To assist with compounds identification and samples needed for activity testing two of the compounds were able to be prepared. Compound 1 is prepared from ascorbic acid and tryptophan, while compound 3 is prepared from tryptophan and acetaldehyde. The ascorbic acid reaction probably involves initial conversion of the ascorbic into an aldehyde and gives low yields. The reaction with acetaldehyde proceeds more rapidly. Compound 2 might be formed from hydroxymethylfurfural (HMF) but would then require a further methylation step.

The preparation of compounds 1 and 3 were performed at a small scale and provided enough material to confirm that the synthetic compounds matched the natural compounds and were also used to optimise the MS analysis in the LCMS. The prepared compounds are also prepared and used in Example 2.

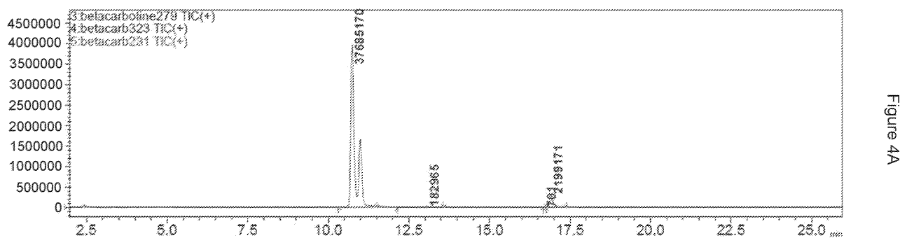

FIG. 3 shows a chromatogram of samples of test drink containing BC. Only the channels for the three betaCs are shown, the blue trace is compound 3 while the peak around 17 min is for compound 1 and the weaker peak at around 13 mins is compound 2. We were not able to quantify these compounds but clearly compound 3 is showing a strong response in this analysis.

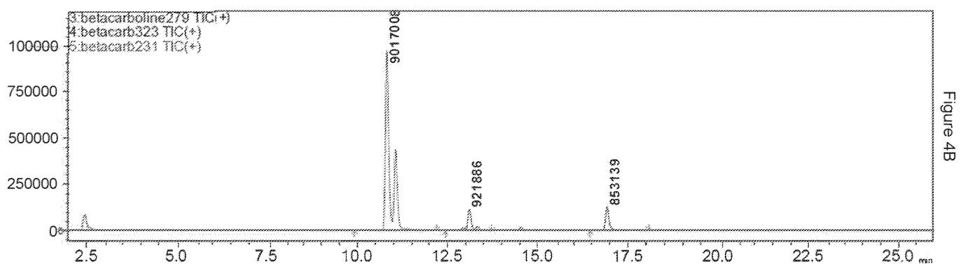

With this method we could then look at other relevant samples. Three samples of frozen Neuroberry™ blackcurrants were supplied for analysis. Juice from a few berries of each was also pressed out, centrifuged and analysed after 1:1 dilution with water. Chromatograms of the Neuroberry™ samples are shown in FIG. 4. This shows a reasonably high level of variation in the amounts of betaCs in the three berry juice samples. For example, the compound 3 peak area was 3.8, 9.0 and 4.9 (relative peak areas in millions of units).

Samples of frozen BC juice concentrate and a dried BC extract powder were also analysed. The BC powder was a dried ethanolic extract of BC. Both the dried ethanolic concentrate and the frozen juice concentrate contained the three betaCs identified above.

### Comparison of Fresh Juice and Dried Extract

As noted above, both the fresh juice and the dried ethanol extract contained betaCs.

However, there are clear differences between the juice and the ethanol concentrate. The most obvious is the absence of polar components in the ethanolic extract. This is to be expected as polar compounds such as sugars and food acids are less soluble in ethanol. Two compounds that are of particular interest for this work are ascorbic acid and tryptophan. Tryptophan is the precursor of the betaCs discussed above and the betaCs are known to form in juice and most probably during processing of juice. Ascorbic acid is also important in this context as some of the betaCs are formed from the reaction of ascorbic with tryptophan.

Analysis of the dried ethanolic extract showed no ascorbic acid present and moderate levels of tryptophan. High levels of ascorbic acid and tryptophan were seen in both the juice and juice concentrate. The actual levels of these components have not been determined.

### Summary

We have developed a method to screen for low levels of specific beta-carbolines in blackcurrant juice products. Three compounds have been identified in fresh juice, juice concentrate and dried ethanolic extract.

Two of the identified beta-carbolines have been prepared from tryptophan combined with ascorbic acid or acetaldehyde. These types of compounds have been demonstrated to form in juices and some fruit during processing.

The ethanolic extract of blackcurrant shows absence of ascorbic acid. This absence of ascorbic and other polar compounds may reduce the formation of beta-carbolines in vivo.

The inventors concluded more detailed analysis may be needed to determine the in vitro MAO activity in juice and samples enriched in betaCs.

### Example 2—Bioassays

In previous Example 1, we identified some betaCs in BC samples and we also prepared some betaCs enhanced samples in preparation for our bioassays.

Most commercial MAO assay kits work by measuring hydrogen peroxide formation (produced when MAO deaminates a monoamine substrate). This method can suffer interference when antioxidants are present in the sample (antioxidants can inhibit the peroxide formation). Work by Herraiz et al (2018) has shown that an alternative direct analysis of the deaminated substrate using HPLC is a viable option. Using this method, they showed common food phenolic compounds such as quercetin and cyanidin are not active (rather they inhibit the peroxidase used in the commercial assay).

In Example 2, the inventors:
used an in vitro assay using a commercial kit to test for MAO-B and possibly MAO-A inhibition;
used the HPLC assay method to confirm activity in various samples including BC juice, dried ethanol extract (35% anthocyanins), and test betaCs produced in Example 1.

### Methods and Results

In this Example we have focused on in vitro evaluation of the effect of BC juice using commercial-kit-based MAO assays.

The kit used was from Sigma-Aldrich [MAK295/296] for the MAO-A and MAO-B enzymes. The kit method detects peroxide formation (resulting from MAO activity) via production of a fluorescent product. As the BC samples are complex mixtures containing some fluorescent components and a high colour background, only a limited number of samples were able to be assayed with each kit (due to the need for multiple controls and background samples).

In the first kit assay run we looked at samples of freshly extracted blackcurrant juice, the dried extract, a test drink containing blackcurrant juice (aged) and a mix of two of the β-carboline samples prepared in the Example 1 (Compounds 1 and 3).

Results are shown in Table 1 below. The values in the table in mg/ml are approximations of the juice content based on anthocyanin comparisons. This was determined by measuring the anthocyanin concentration of the dried extract and the juice concentrate (UV-Vis) relative to the fresh squeezed juice. So, the juice concentrate is 4.5× the concentration of the juice and the dried extract is 73× the concentration of the juice. The test drink beverage is known to be a 1:1 dilution of the juice.

TABLE 1
Results from the first round of assays

| Sample description | MAO-A inhibition | MAO-B inhibition |
|---|---|---|
| Neat Juice (10 × dilution) (100 mg/ml) | 93.25% | 86.46% |
| Neat Juice (100 × dilution) (10 mg/ml) | 40.88% | 38.21% |
| Dried extract at 2.5 mg/mL (182.5 mg/ml) | 99.33% | 100.22% |
| Dried extract at 0.25 mg/mL (18.25 mg/ml) | 83.93% | 76.42% |
| Juice concentrate at 5 mg/mL (22.5 mg/ml) | 100.00% | 179.34% |
| Neat drink at 20 × dilution 25 mg/ml | 76.39% | 119.90% |
| Neat drink at 2 × dilution 250 mg/ml | 101.91% | 101.02% |
| βcarbolines (mix of 2) at 0.05 mg/mL | 8.42% | 0.46% |
| βcarbolines (mix of 2) at 0.025 mg/mL | 2.58% | 0.00% |
| Inhibitor control for MAO-A (Clorgyline at 5 uM) | 94.16% | >100% |
| Inhibitor control for MAO-B (Selegiline at 5 uM) | >100% | 97.82% |

These results were very encouraging. We saw a good MAO-A and MAO-B inhibition with the neat juice sample and juice concentrate. We also see inhibition with both the dried extract and the test drink product.

However, we see little inhibition with the β-carboline mix. While this latter result is somewhat disappointing it is only two from a range of possible betaCs and published work shows huge variability in betaC activity according to structure.

In general, both MAO-A and MAO-B inhibition is seen for the active samples with optimal testing rate around 10 mg/ml juice equivalent.

A second round of kit testing was undertaken (Table 2, below). For this round the juice was retested at three dilutions, namely 20, 10 and 5 mg/ml. These samples show MAO-A results consistent with the previous set, with good on-scale activity at 5 and 10 mg/ml juice equivalents.

Because of the ongoing concern that the activity was aligned with the flavonoid/anthocyanin polyphenols in the juice, we also tested a series of fractions which were attempts to separate the polyphenols and the activity, with results shown in Table 2.

Extracts E1, E2 and E3 are samples prepared by diethyl ether extraction of juice concentrate (diluted in water). E2 is an ether extraction of E1 after it was made alkaline, and E3 is an ethyl acetate extraction of E2.

None of these extracts were active, indicating that the activity is not associated with a non-polar part of the extract and so is unlikely to be associated with the known β-carbolines.

Another way to separate the polyphenols from “other” components is to pass the extract through a polyamide column. The results of this are also shown in Table 2 below. In this case, a small sample of juice (from concentrate) was made up in 0.25% acetic acid and loaded onto a polyamide column, PA1 is the load fraction, PA2 is the polar non-phenolic fraction eluted with the 0.25% acetic acid, PA3 is the main polyphenol fraction (coloured) eluted with 1:1 methanol: water while PA4 is eluted with neat methanol. The most activity is seen in fraction PA2 which is the polar material separated away from the main anthocyanin/polyphenol fraction (PA3). This tells us the primary activity surprisingly is not associated with the anthocyanins/polyphenols and not β-carbolines, as was expected.

TABLE 2
Results from the second round of assays.

| | MAO-A | MAO-B |
|---|---|---|
| Sample description | inhibition | inhibition |
| Neat Juice (50x dilution), 20 mg/mL | 100.00% | >100% |
| Neat Juice (100x dilution), 10 mg/mL | 54.13% | >100% |
| Neat Juice (200x dilution), 5 mg/mL | 45.41% | >100% |
| Extract J10 E1, 100 × D in water | 14.22% | 32.86% |
| Extract J10 E2,100 × D in water | −11.40% | 12.86% |
| Extract J10 E3, 100 × D in water | −21.07% | 9.72% |
| Fraction J10 PA1, 100 × D in water | 43.45% | >100% |
| Fraction J10 PA2, 100 × D in water | 86.57% | >100% |
| Fraction J10 PA3,100 × D in water | 25.85% | 51.43% |
| Fraction J10 PA4, 100 × D in water | 36.12% | >100% |

In this second round of assays, the MAO-A results have generally been on-scale, however the MAO-B is seemingly more sensitive and most results were >100%. However, we still see MAO-B activity consistent with MAO-A suggesting we need only perform one of the assays to follow the activity in subsequent Examples.

### Summary

Activity testing using the Sigma-Aldrich commercial kit has shown consistent MAO-A and MAO-B activity for the juice, juice concentrate and dried juice extract. The activity is concentration dependent. The MAO-A and MAO-B activity appears to track together so only one (MAO-A) assay is suggested for future Examples, for efficiency.

Testing of some fractions which separated the polar and non-polar components from the main juice polyphenols showed that activity is associated with a polar non-polyphenolic fraction from the juice. This suggested the activity was not due to known β-carbolines as they would be concentrated in the non-polar fractions. This work leads into Example 3 which will aim to identify the specific fraction and component(s) that are responsible for the MAO inhibitor activity.

### Example 3: Bioassay Guided Fractionation

### Overview

In Example 3, the inventors looked at fractionation processes to try to narrow down the identity of the active component(s).

### Methods and Results

To allow for screening of larger numbers of fractions/samples, an alternative assay was used in Example 3 to the Sigma-Aldrich commercial assay kit used in Example 2. We used a porcine S9 liver microsome fraction as the source of the MAO-A and MAO-B enzymes. This method allowed the screening of many more samples than possible with the kit-based assays. A S9 pig liver microsome was isolated from macerated pig livers.

The S9 porcine fraction was mixed with a specific MAO substrate (kynuramine) and mixed with the test sample and buffer. After 1 hr at 37° C. the reaction was halted by cooling and addition of 2N NaOH and acetonitrile. The centrifuged sample was then analysed using LCMS and the level of 4-quinolol (4HQ) product measured.

The method was performed according to the protocol described in Ghosal., (2020), entitled “Evaluation of the clearance mechanism of non-CYP-mediated drug metabolism and DDI as a victim drug.” This method takes advantage of the spontaneous formation of 4-quinolol from the oxidised kynuramine. The levels of kynuramine and 4-quinolol are assessed by LCMS and the low level/absence of 4hydroxyquinolol indicates inhibition of MAO.

This method proved to be very useful for following the MAO inhibition activity through a number of separation steps.

### Preliminary Separation Using Column Chromatography (CC).

Three modes of CC were used.

### 1. Reversed Phase Chromatography (RP CC)

Initially, a small-scale column was run with a mini-RP column, eluted with water followed by increasing proportions of methanol.

The MAO inhibition activity was only seen in the most polar fractions, i.e. those eluted at the start.

Following this a larger sample of the juice concentrate (5 g) was dissolved in water and pH adjusted with 2N NaOH to pH 5.5. This extract was applied to a bigger C18 silica RP column and eluted with water (two fractions) followed by acidified water (2 fractions) followed by elution with 15% ethanol in water and finally ethanol.

Assay of these larger fractions showed the main activity to be confined to the two initial water fractions.

### 2. Cation Exchange Chromatography

Small scale chromatography using commercial pre-prepared column (SPE type). A sample of juice concentrate was dissolved in pH 8 buffer and loaded onto the column. The column was then rinsed with 0.1N HCl followed by water and then NaOH.

The MAO inhibition activity was concentrated in the load fraction, i.e. was not retained on the column.

### 3. Anion Exchange Chromatography

Several trials were performed using anion exchange material. Initially it appeared that the activity was retained on the column and eluted with acid. However, the assay proved to be somewhat sensitive to traces of acid so when samples were completely dried and made up in water, the activity was confined to the load fractions.

These results demonstrated that the active material in the BC juice concentrate was polar (eluted early on RP CC) and was neither acidic nor basic in nature (was not retained on anion or cation exchange media).

### Preparative HPLC

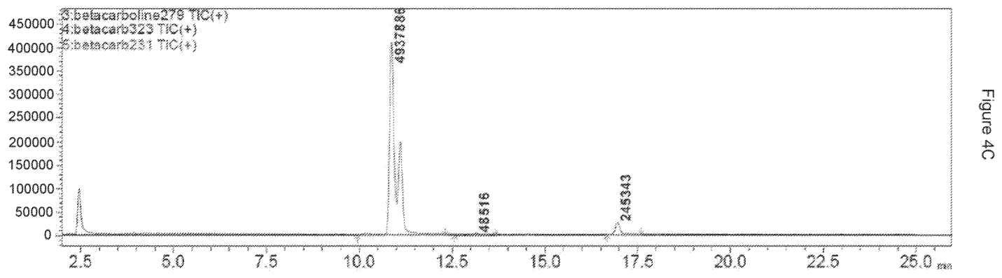

Preparative HPLC was the next step to further narrow down the activity. Firstly, a chromatographic run was performed using a 250×10 mm Luna C18 column. Fractions were collected every minute and a gradient of acetonitrile in acidified water was used. Subsamples of each fraction were taken and dried before assay. The assay profile is shown in FIG. 5. As shown, the activity is confined to the early fractions in the HPLC run, mainly in fraction 3 and 4 but also in 8.

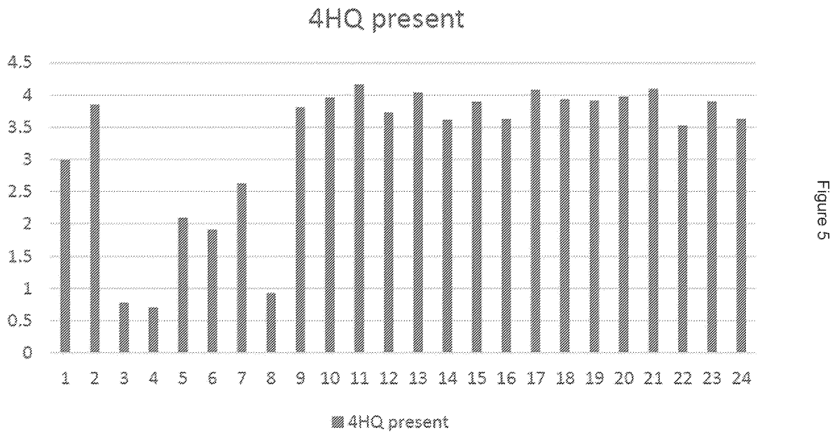

To narrow down the activity further a second preparative HPLC run was performed (see FIG. 6). This used a larger column (Supelco Ascentis C18, 250×22 mm) and more fractions were collected around the area of interest (Fractions 18-23 were collected from 7-11 minutes).

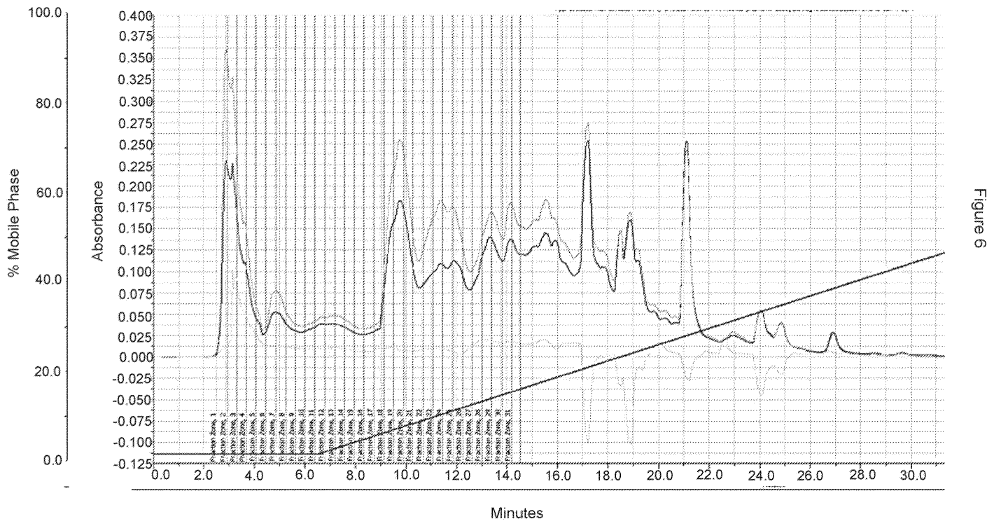

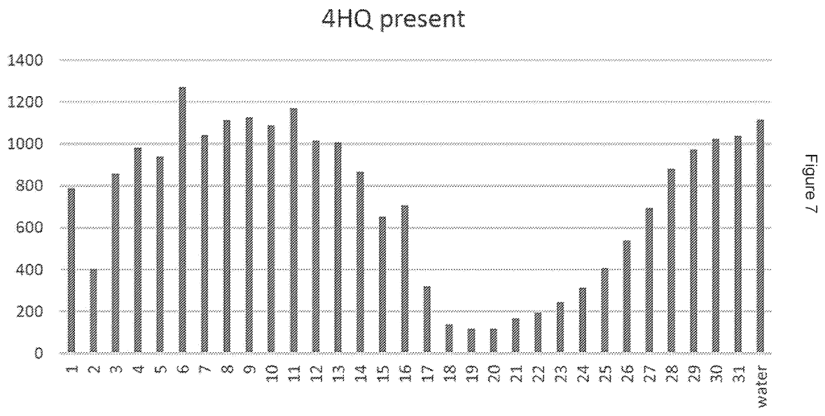

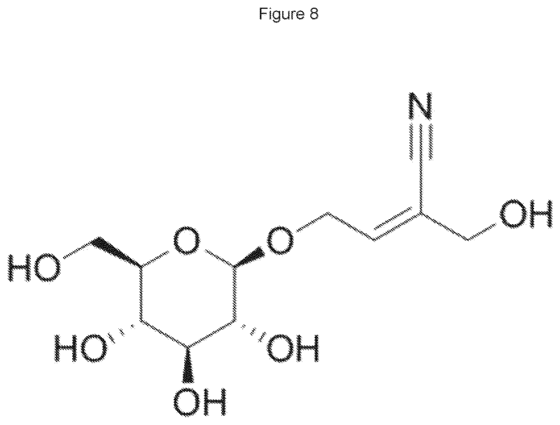

As shown in FIG. 7, the most active fractions in the assay were 19 and 20. LCMS analysis indicated these fractions were probably a single compound with a molecular weight of 276. High resolution MS indicated a formula of C₁₁H₁₇O₇N. The main ion seen in negative mode is a formic acid adduct (m/z 320). Fractions 19 and 20 were combined and NMR spectra obtained. This NMR data indicated the presence of a sugar (likely glucose). A search of the literature using the identified formula and presence of a sugar enabled the most likely candidate to be identified as sarmentosin (shown in FIG. 8), a nitrile glycoside previously isolated from plants such as Kalanchoe species. Comparison of NMR data with that for sarmentosin confirmed this assignment.

To the best of the inventor's knowledge, there has never been any identification or discovery of MAO activity from sarmentosin. Therefore, the present invention relates to the new use of sarmentosin as a MAO inhibitor (and associated uses thereof as described elsewhere in this specification) from a natural source for example could be from the likes of blackcurrant or Kalanchoe species, or derived in other ways such as through cell-based fermentation and purification technologies, or synthetic production of the compound.

Sarmentosin can be classified as a gamma hydroxy nitrile glycoside. This is an unusual class of hydroxy nitrile glycosides as most known hydroxy nitrile glycosides are alpha hydroxy nitrile glycosides (e.g. prunasin from apple seeds).

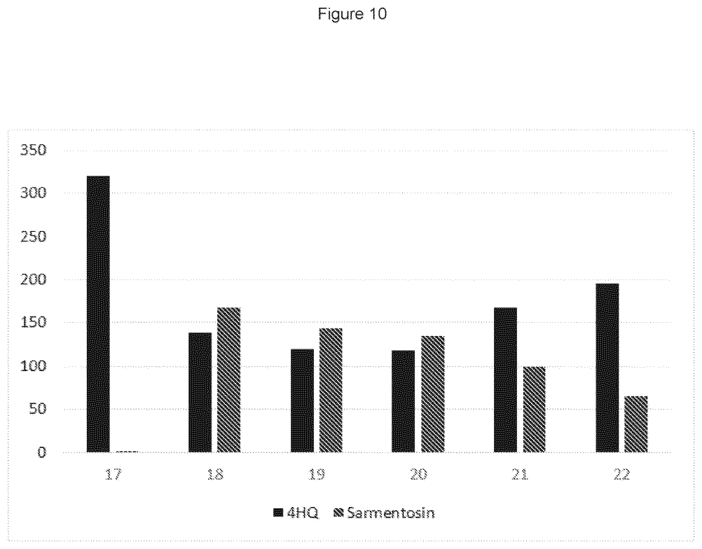

As shown in FIG. 10, the further HPLC analysis of the group of most active fractions appears to show the highest levels of sarmentosin in fractions 18-21. This appears to fit with sarmentosin being the active component.

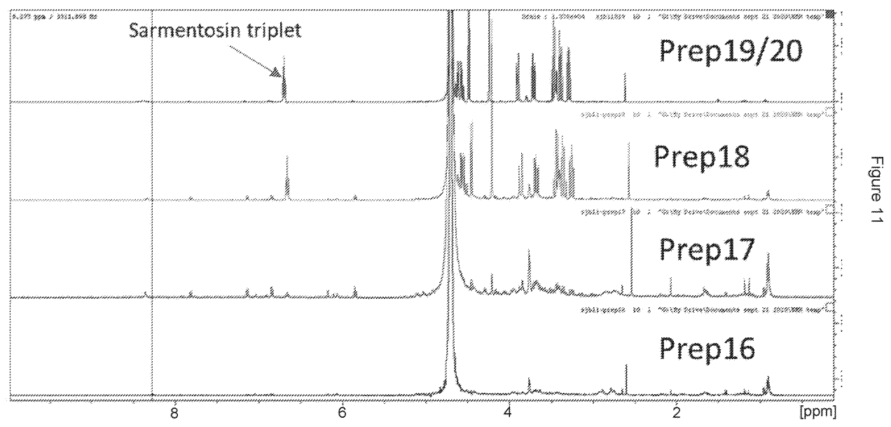

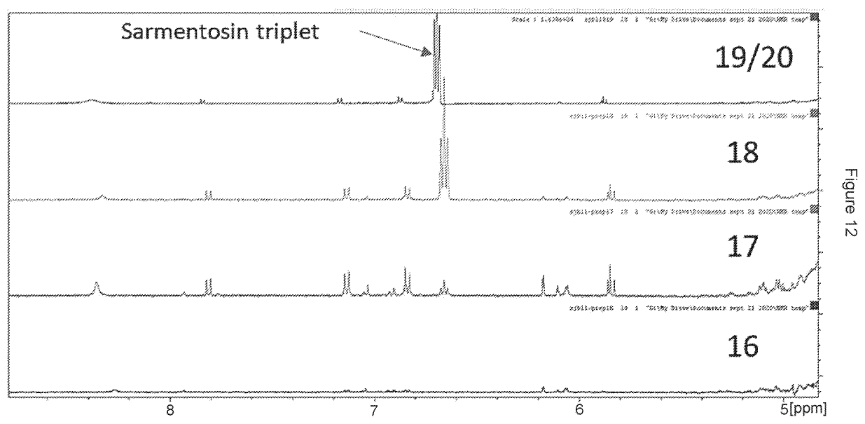

We also closely examined the NMR spectra of the fractions just before and those containing sarmentosin (see FIG. 11). Fraction 19 and 20 combined show dominant NMR signals for sarmentosin and only tiny amounts of other compounds. Fraction 18 is similar with a few small peaks in the 5.5-9 ppm region. Fraction 17 has a small amount of sarmentosin while the other peaks in the 5.5-9 ppm region are larger (detail in FIG. 12). Fraction 16 (inactive) has no sarmentosin and is also missing the other signals in the 5.5-9 ppm region. This is consistent with sarmentosin being the source of the activity in these fractions.

### Revisiting Column Chromatography Fractions.

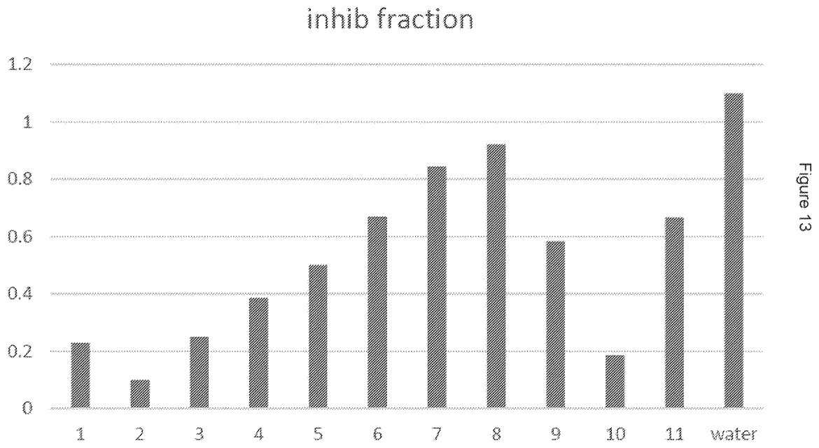

With the likely activity in the polar fractions identified we decided to check for any other activity in fractions from a reversed phase C18 column. A fresh column was run using 5 g of concentrate and 11 fractions were collected. When these fractions were assayed using the S9-LCMS method a second band of activity was observed in the late eluting non-polar fractions (See FIG. 13). Fraction 10 is active. The main sarmentosin fraction is Fraction 2.

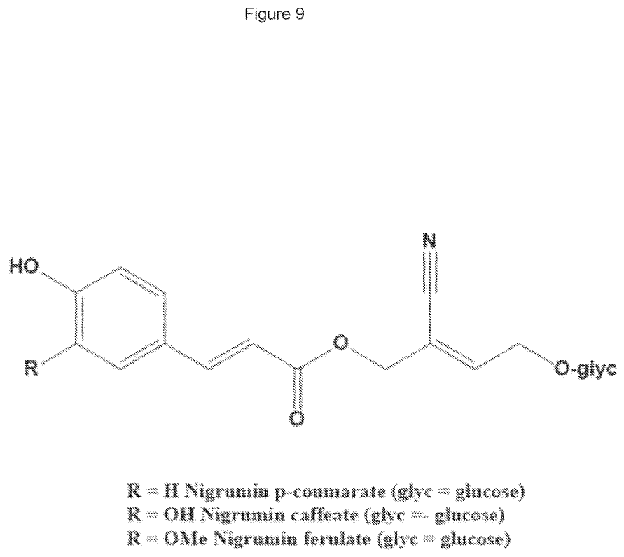

Analysis of fraction 10 shows the presence of a set of flavonol glycosides as well as some other phenolics. Among these phenolics, we identified the presence of the two nigrumin ester derivatives of sarmentosin as per FIG. 9) through LCMS extraction of ions. These two related compounds, although again not identified as having MAO-inhibition activity, have been previously isolated from blackcurrant seeds (Lu et al., 2002). These are nigrumin-p-coumarate and nigrumin ferulate (FIG. 9). They are the phenolic acid derivatives of sarmentosin.

A sample of fraction 10 was subjected to RP prep HPLC and fractions were assayed using the S9 assay as used previously. The results (shown in Figure x) indicate three bands of activity, fraction 9/10, fractions 12-16 and a late band (fraction 21).

These active fractions were analysed using LCMS (as above). The coumaroyl and feruloyl appear to be concentrated in the fractions 12 to 16 (Figure x, peaks areas vs fraction number). This is consistent with the activity shown in the band of fractions. Fractions 9 and 10 shown also showed some characteristic mass spectral peaks to indicate the presence of a further sarmentosin ester. This was determined to be a caffeoyl ester of sarmentosin with a characteristic MRM transition (m/z 436->179). This appears to be the compound responsible for the observed activity in fractions 9 and 10 (FIG. x).

Note the HPLC fraction are mainly mixtures of compounds. There are high levels of other components, mainly flavonoids, in many of these fractions but especially fraction 8-12.

Fractions 13 and 14 are dominated by the ester compounds.

Through access to a purified sample of the two nigrumin esters from historic work and an S9-LCMS assay, the nigrumin mixture was active at about the same level as the sarmentosin. This strongly supports that the active compound in the S9 assay is sarmentosin. The sarmentosin esters (nigrumin ferulate/coumarate) are possibly hydrolysed to yield sarmentosin by the S9 enzymes.

### Comparison of Extracts

In the human studies the ethanolic extract powder (DelCyan) was less effective at altering MAO activity. In this work the inventors have analysed the provided blackcurrant powder and found very low levels only of sarmentosin (compared to juice or juice concentrate). This also is evident in the S9 LCMS bioassay where the juice concentrate is more active than the dried extract when compared at the same level of total anthocyanins (dried extract 73×juice, concentrate 4.5×juice). The dried extract would be expected to still contain the nigrumin esters.

### Summary

In conclusion:
MAO inhibition activity appears to be tracked primarily to sarmentosin and to a lesser extent the sarmentosin esters.
Activity is most readily observed using a crude liver S9 fraction containing a mix of enzymes.

### Example 4: Further Analysis of Sarmentosin Activity in Samples

### Overview

In Example 4, samples of the active compounds were purified from the juice concentrate (scale up of work from Example 3) to give ca. 10 mg of each active compound. Furthermore, juice or other berry samples (e.g. blueberry, and various blackcurrant samples) are analysed for the content of these actives Finally, the ester-containing fraction was subjected to fractionation and bioassay (S9 assay) to confirm activity for esters.

### Results

### 1. Purification of Active Compounds.

Approximately 2 kg of frozen berries were blended with an equal volume of 1:1 ethanol: water. The liquid was separated from the remnant solids using a fine nylon cloth as a filter. The ethanol was removed from the filtrate by rotary evaporation. Half of the liquid extract was then applied to a reversed phase chromatography column plug (dimensions 9 cm diameter and 4 cm deep). After loading the column was eluted with water containing 0.1% formic acid and eluted with increasing proportions of ethanol in water.

The fractions were analysed using LCMS. The sarmentosin was concentrated in the fractions eluted with water and up to 5% ethanol while the sarmentosin esters were found in the fraction eluted between 40 and 60% ethanol. The rest of the extract was chromatographed in a second run.

Fractions containing sarmentosin were combined, freeze dried and re-chromatographed using the same column. The sarmentosin-rich fractions from this separation were then used for preparative HPLC. Preparative HPLC was performed using a Gilson prep system, elution with acetonitrile and 0.1% formic acid in water using a gradient beginning with 1% acetonitrile and ending with 15% acetonitrile. The purity of the sarmentosin fractions were assessed using LCMS and 1H NMR.

Similarly, the sarmentosin ester rich fractions were combined, re-chromatographed using the RP plug and finally purified using preparative HPLC (same system as above but using a modified gradient, 5 to 40% acetonitrile). The purity of the esters was assessed using LCMS.

### 2. Analysis of Samples.

Four comparison blackcurrant juice drinks were used, which we have labelled as Samples 1-4, and compared to the Applicant's test blackcurrant juice sample, provided as a pure Neuroberry™ blackcurrant juice extraction (Sample 5), and also in a concentrated juice (Sample 6), and commercial form (Sample 7).

Furthermore, we tested two extracts from freeze-dried blackcurrants (Samples 8 and 9), a useful comparison to the Applicant's test dried extract containing blackcurrant material (Sample 10).

The samples were analysed using LCMS. Standards were prepared for sarmentosin, feruloyl sarmentosin and coumaroyl sarmentosin. Analysis used specific mass data (MRM's) for each of the analytes and peak areas for the samples were compared with those for the standards.

The juices were analysed as neat or diluted 1:1. The freeze-dried berries were extracted with water or ethanol: water (1:1).

The values were then compared with values obtained for Samples 5-7.

The results are shown in Table 3. Note the sarmentosin content is shown in mg/g of sample while the esters are ug/g. From this data we can see that Sample 5 has about 15× as much sarmentosin as each of the esters. The amount of the two esters is similar.

Based on these initial results, the inventors consider a typical level of sarmentosin in a different types of blackcurrant juices/extracts may range substantially from about 5 to 1300 μg/g. Therefore, in one aspect of the invention, the inventors foresee using a juice extract or other format with sarmentosin or ester(s) either within this range for the new use towards MAO inhibition, or advantageously increased above this level to increase the potency or effectiveness of a composition, nutraceutical or extract.

We can also conclude that the freeze-dried berry extract samples 8 and 9 have approximately the same content as Sample 5. Juice sample 2 is roughly similar to Sample 5 although the sarmentosin level is somewhat lower. In juice sample 4, sarmentosin content is consistent with a 5.7% juice content. The test drink product 7 was from an aged sample stored at room temperature so may have lost some of the sarmentosin over time. This test drink is juice at 1:1 dilution so it would be expected to normally contain about 0.7-0.8 mg/g sarmentosin in a fresher sample. The concentrated beverage sample 6 and dried powder (Sample 5) are both very high in sarmentosin compounds however the dry extract has proportionately lower levels of sarmentosin (esters are about three times the level in Sample 6 but sarmentosin is lower). This is likely the effect of the use of ethanol which would favour the extraction of the less polar esters.

TABLE 3
Analysis of sarmentosin and esters content in various juice products.

| | Sarmentosin | Coumaroyl | Feruloyl |
|---|---|---|---|
| Sample | (μg/g) | ester (μg/g) | ester (μg/g) |
| Blackcurrant juice 1 (Sample 1) | 22.0 | 4.9 | 3.2 |
| Blackcurrant juice 2 (Sample 2) | 80.0 | 114.7 | 98.7 |
| Blackcurrant juice 3 (Sample 3) | 16.0 | 1.9 | 1.2 |
| Blackcurrant juice 4 (Sample 4) | 6.0 | 3.8 | 2.5 |
| Test blackcurrant juice - freshly extracted | 225.0 | 108.0 | 64.8 |
| (Sample 5) - Neuroberry ® | | | |
| Test blackcurrant juice - concentrated | 1277.0 | 1116.6 | 1220.2 |
| (Sample 6) | | | |
| Test blackcurrant juice 1:1- neat and aged | 22.0 | 21.7 | 14.0 |
| (Sample 7) | | | |
| Ethanol/water extract of freeze-dried | 114.0 | 118.3 | 61.3 |
| blackcurrants (Sample 8) | | | |
| Water extract of freeze-dried blackcurrants | 134.0 | 94.0 | 67.0 |
| (Sample 9) | | | |
| Test blackcurrant dried extract composition | 1096.0 | 3092.2 | 3209.2 |
| (Sample 10) | | | |

### Fractionation and Bioassay of Ester Rich Fractions

In Example 3, it was noted that a less polar fraction from reversed phase chromatography was also active in the S9 bioassay. This fraction (RP10) showed good activity and appeared to contain the sarmentosin esters discussed above.

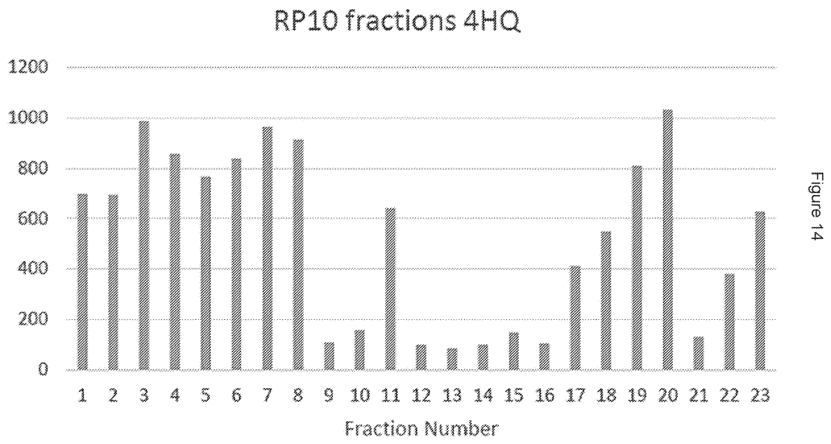

A sample of RP 10 was subjected to RP prep HPLC and fractions were assayed using the S9 assay as used previously. The results (shown in FIG. 14) indicate three bands of activity, fraction 9/10, fractions 12-16 and a late band (fraction 21).

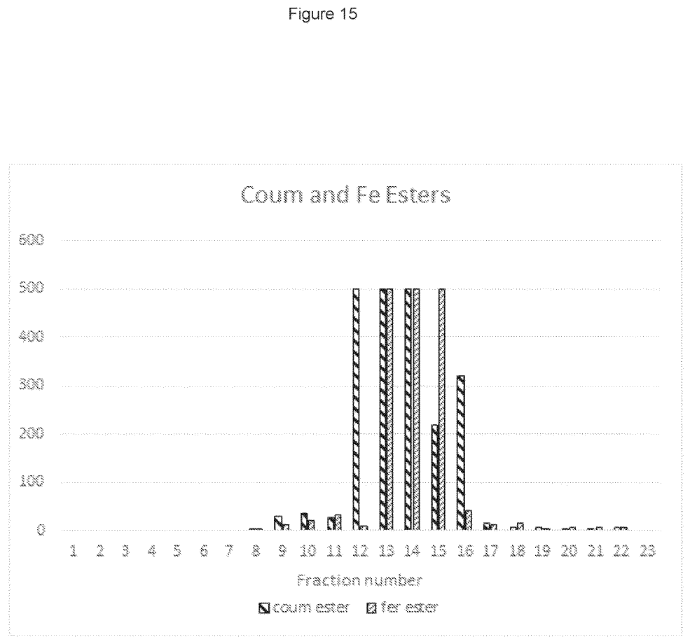

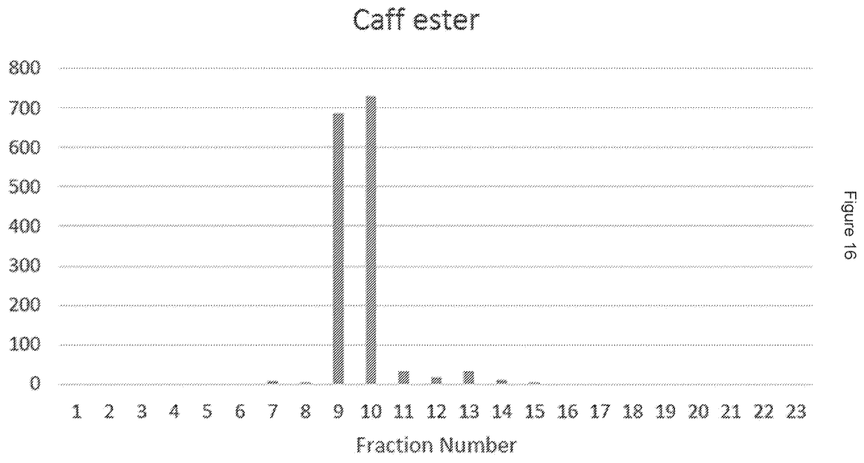

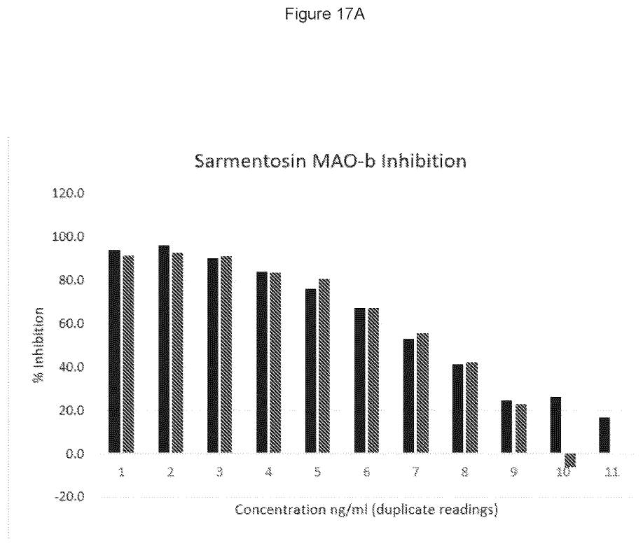

These active fractions were analysed using LCMS (as above). The coumaroyl and feruloyl appear to be concentrated in the fractions 12 to 16 (FIG. 15, peaks areas vs fraction number). This is consistent with the activity shown in the band of fractions. Fractions 9 and 10 shown also showed some characteristic mass spectral peaks to indicate the presence of a further sarmentosin ester. This was determined to be a caffeoyl ester of sarmentosin with a characteristic MRM transition (m/z 436->179). This appears to be the compound responsible for the observed activity in fractions 9 and 10 (FIG. 16).

Note the HPLC fraction are mainly mixtures of compounds. There is a high level of other components, mainly flavonoids, in many of these fractions but especially fraction 8-12. Fractions 13 and 14 are dominated by the ester compounds. The later active fraction (22) did not show any peaks in the LCMS to differentiate it from fractions 20 or 22.

### Conclusions

This work on the blackcurrant juice has shown that sarmentosin is present at relatively high levels in juice samples. Low levels of phenolic esters of sarmentosin are also present and these are also active. Coumaroyl and feruloyl esters are the main esters but the caffeoyl ester has also been identified in active fractions.

### Example 5: Comparison of Sarmentosin MAO-B Activity Vs Deprenyl

### Overview

This example compares MAO-B activity of sarmentosin to Selegeline (also known as Deprenyl or L-deprenyl, and sold under brand names such as Eldepryl and Emsam is a known MAO-B inhibitor and is used to treat Parkinson's disease, and major depressive disorder). This helps to validate effective dosages of sarmentosin alone, or for example within an extract (such as a juice).

### Results

Deprenyl was purchased from Sigma-Aldrich. (R-(−)-deprenyl, this compound is also known as selegiline). The sample of sarmentosin was from material purified in the earlier work. The concentration of the sarmentosin was confirmed using NMR with a quantitative NMR standard. The S9 liver microsome fraction containing the crude enzyme mix was from a frozen bulk sample derived from porcine liver.

Samples were prepared in a 6-well plate (deep well format with 1.1 ml strip tubes). The assay was performed by diluting the crude S9 fraction with buffer (PBS, PH 7.4, diluted 1 in 5, 150 μl) and adding the test sample dissolved in buffer (75 ul). An aliquot of kynuramine was added (10 μl of 1 mg/ml) and the plate was placed in a 37 C water bath for 1 hr. The plate was cooled in ice water before addition of 20 μl 2N NaOH followed by 250 μl acetonitrile. The individual strip tubes were centrifuged, and samples taken from each for LCMS analysis.

The LCMS analysis was performed using a Cyano HPLC column eluted with 0.1% formic in water and acetonitrile. The LC run was isocratic mode (40% Acetonitrile) and the relative levels of 4 hydroxy quinoline (4HQ) and kynuramine determined from peak areas. The % inhibition was determined by comparing the amount of 4HQ with that of a buffer-only sample well. Kynuramine is converted to 4HQ by the MAO-B enzyme.

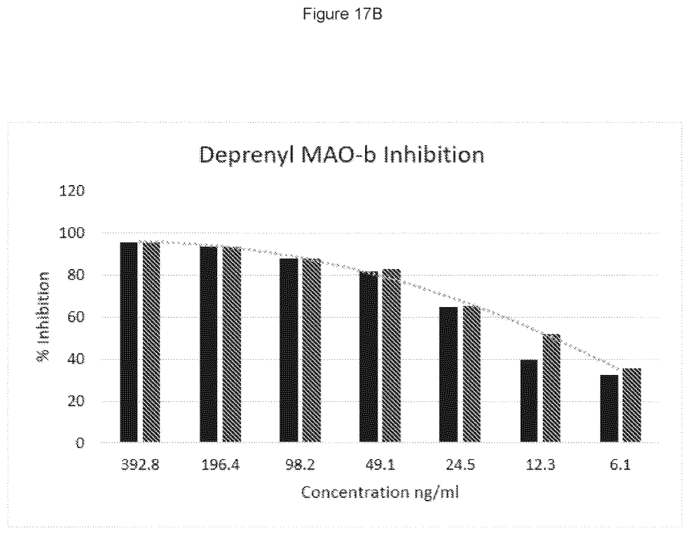

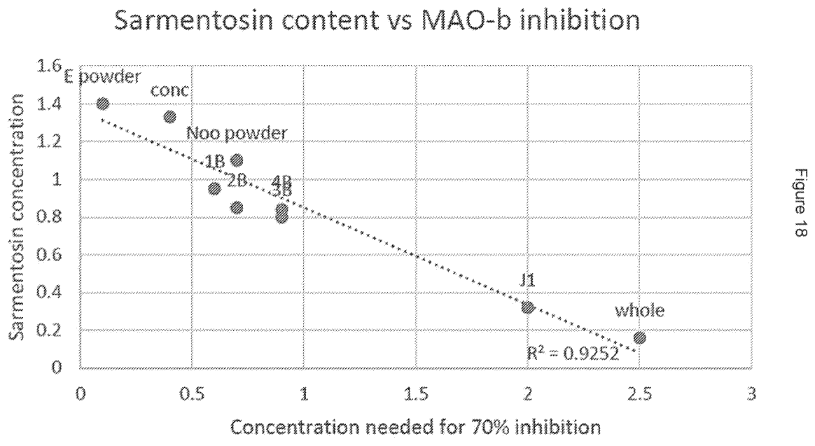

The results of this analysis are described below, with reference to FIG. 17.
The sarmentosin 50% inhibition is seen at around 400 ng/ml, which is equivalent to a concentration of 1.45 μM.
The Deprenyl 50% inhibition is seen at around 15 ng/ml, or 67 nM.
This indicates that Deprenyl is a 22-fold stronger inhibitor of MAO-B than sarmentosin.
A recommended minimum starting dose of Deprenyl (selegiline) is 1.25 mg/day and increased to 2.5 mg/day after 6 weeks (see https://www.drugs.com/dosage/selegiline.html).

### Discussion:

Based on these results, a preferred dosage of sarmentosin for MAO-B inhibition for cognitive diseases or preventative/improvement in mental health is at least about 27500 μg sarmentosin/day.

Therefore, for a blackcurrant juice that includes for example 320 μg/g sarmentosin, a daily serving of 86 ml of this blackcurrant juice is likely to provide about 27500 μg sarmentosin which may provide particularly preferred efficacious dose when used to achieve MAO-B inhibition (i.e. when applying the 22× conversion, would be equivalent to an efficacious dose of 1.25 mg Deprenyl).

### Example 6: Analysis of Sarmentosin Content/MAO Activity in a Range of Blackcurrant Berries, Juice and Juice Concentrates

### Overview

This example assesses a range of blackcurrant cultivars for both MAO activity (S9 assay as above) and sarmentosin content (LCMS analysis).

### Results

As part of ongoing improvements to the analysis method the LCMS method employed for the previous work was adapted. The previous method suffered from an overlap of metabolites eluting around the same time as sarmentosin. This led to interactions between the metabolites giving suppression of the sarmentosin signal and hence incorrect estimations of concentrations. The HPLC method was improved by the use of 10 mM ammonium formate in the aqueous solvent (instead of 0.1% formic acid). This change moved the sarmentosin peak to elute later than the food acids and sugars and gave more consistent results for the samples and standards.

The sarmentosin standard was also reassessed as part of this work. Previously the standard was the sample of sarmentosin isolated from preparative HPLC and shown to be “pure” using NMR.

However, when the purity was assessed using a quantitative NMR standard the sample was found to be of lower purity. All subsequent analysis is based on this new NMR standard.

Various juice samples, concentrates, powders and whole fruit were tested. Each of the samples was diluted with water to give a sarmentosin peak that was around the appropriate concentration for the LCMS analysis. From the results from Example 5 we could estimate the approximate concentration of sarmentosin required to give a 20-80% inhibition in the S9 assay. The samples for the assay were diluted to this approximate concentration by dilution with water and the assay performed using 4 wells for each sample in a 2× dilution series. Due to constrictions of supply of reagents the assays were only performed once for each sample.

The results are shown below in Table 4, with the amount of sarmentosin in each sample and then the concentration of sample in mg/g which gives either 70 or 80% inhibition in the assay.

The ethanolic extract is a powder which is an ethanolic extract of the berries. The whole berry sample is a water extract from whole berries. The Viberi® sample is a commercial sample of packaged freeze-dried whole berries.

### Discussion

These results are consistent with the sarmentosin being the effective active in the samples. FIG. 17 is a plot of the concentration needed for 70% inhibition vs the sarmentosin concentration. This indicates a direct correlation between the sarmentosin content and the inhibitory activity.

TABLE 4
Sarmentosin and relative MAO-B inhibition activity
for a range of blackcurrant samples.

| | Sarmentosin | 70% Inhibition | 80% Inhibition |
|---|---|---|---|
| Sample | (mg/g) | (mg/g) | (mg/g) |
| Blackcurrant Juice (Neuroberry) | 0.32 | 2 | 3 |
| Blackcurrant concentrate (65 Brix) | 1.33 | 0.4 | 0.8 |
| Blackcurrant powder | 1.1 | 0.7 | 0.9 |
| Blackcurrant Juice (Neuroberry blend | 0.12 | 7.5 | 14.0 |
| 1:3) | | | |
| Ethanolic Powder | 1.41 | 0.1 | 0.5 |
| Concentrate 1B | 0.95 | 0.6 | 1.5 |
| Concentrate 2B | 0.85 | 0.7 | 1.3 |
| Concentrate 3B | 0.8 | 0.9 | 1.5 |
| Concentrate 4B | 0.84 | 0.9 | 1.5 |
| Whole blackcurrant (water extract) | 0.16 | 2.5 | 6.0 |
| Viberi ® FB Berry (freeze-dried) | 0.36 | Not tested | Not tested |

### Example 7: The Effects of Consuming a Single Dose of Blackcurrant Juice or Freeze-Dried Powder on Phytochemical Bioavailability, Platelet MAO-B Activity, Plasma Neurotransmitter Concentrations and Mood in Healthy Adults

Commissioned by Alphagen NZ Limited, Plant and Food Research Limited conducted a randomised, double-blind, two-arm, placebo-controlled, cross-over human intervention study to evaluate temporal platelet monoamine oxidase B (MAO-B) enzyme activity, phytochemical bioavailability, circulating neurotransmitters, and subjective markers of mood after participants consumed beverages prepared from two blackcurrant product formats: juice concentrate and freeze-dried powder. Findings from this study showed that:
Anthocyanin bioavailability in plasma was detectable 10 min after consuming the blackcurrant interventions, peaking at 120 min, and then declining at subsequent timepoints. Greater anthocyanin bioavailability was measured in the blackcurrant powder intervention than with the juice concentrate intervention. This is probably due to the higher anthocyanin dose consumed by participants allocated to the powder intervention.
Sarmentosin bioavailability profiles following blackcurrant powder and juice consumption were similar to the anthocyanin profiles. There were no differences in plasma sarmentosin concentrations between formats at any of the timepoints, despite the differences in anthocyanin dose consumed by participants allocated to the blackcurrant powder and juice arms.
Both blackcurrant formats were similarly efficacious in reducing peripheral platelet MAO-B enzyme activity, by −89.6%+2.2 and −90.5%+2.8, respectively. Significant enzyme inhibition was observed as early as 10 min after treatment consumption and persisted up to the final timepoint of the trial day (480 min post consumption) in both juice concentrate and powder formats.
The inhibition of platelet MAO-B by both blackcurrant formats corresponded with significant reductions in circulating concentrations of the monoamine metabolites DOPAC (metabolite of dopamine), 5-HIAA (metabolite of serotonin) and MHPG (metabolite of norepinephrine). A significant reduction in circulating vanillylmandelic acid (VMA) and 3,4-dihydroxyphenylglycol (DHPG) was observed after consuming blackcurrant juice, but not powder, indicating differences in the modulatory effects between the two blackcurrant formats.
Subjective mood data collected during the study suggest the efficacy of both blackcurrant formats in improving alertness and reducing stress and mental fatigue. Better improvements were observed in the blackcurrant powder interventions, which may have been due to the higher doses consumed by volunteers allocated to the powder intervention.

### Background

A previous study comparing the bioactivity of blackcurrant juice and blackcurrant powder suggested the format of delivery (i.e., extracts, freeze-dried, juice) may affect the bioactivity of blackcurrants in supporting cognition. Significant MAO-B inhibition detected following consuming blackcurrant juice was not observed when an anthocyanin-rich extract was consumed, despite participants consuming the same relative polyphenol dose for both interventions (Watson et al. 2015). The difference in efficacy between the two food formats may be attributed to the removal or degradation of critical bioactive compounds during processing steps to produce the extract format. Therefore, food format and processing conditions are important considerations when formulating blackcurrant-based foods for nootropic benefits.

### Aims and Objectives

Primary aim: To characterise the temporal changes in platelet MAO-B enzyme activity after participants consume a single serve of two different blackcurrant formulations.

Secondary aims: To characterise the temporal blackcurrant polyphenol bioavailability, sarmentosin bioavailability, circulating neurotransmitter concentrations and mood parameters after participants consumed a single serve of two different blackcurrant formulations.

### Methodology

### Trial Beverages

This study tested two Neuroberry® blackcurrant (BC) product formats: juice concentrate and freeze-dried powder. Each BC format was compared with a placebo (PL) beverage that was matched as close as possible to the corresponding BC beverage's appearance, flavour and texture. The BC powder and matched PL powder were supplied, ready to use, by AlphaGen Limited. The BC and PL juice concentrates were formulated by The New Zealand Institute for Plant and Food Research Limited (PFR), within its food-safe laboratory, with ingredients supplied by AlphaGen and Sensient Technologies. The BC and placebo formulations were weighed and portioned into single-serve quantities in amber bottles, stored at −20° C., then served to participants as a 300-mL beverage.

The total amount of BC anthocyanins contained within the two BC beverages differed between the two formats, as previously agreed on by AlphaGen and PFR. The anthocyanin dose of the BC powder beverage (BC-P) was standardised to each participant's weight, so that they consumed a weight of BC powder equivalent to 7.8 mg total anthocyanin/kg of their bodyweight, reconstituted in 300 ml water. The placebo powder beverage (PL-P) was an equivalent weight of placebo powder as the BC powder, in 300 ml water. Apple juice concentrate and clove extract were added to both BC and PL concentrate formulations to increase flavour complexity. Citric acid was added to both concentrate formulations, but in greater quantity for the placebo concentrate beverage (PL-JC), to match the sourness of BC-JC. The flavour and colour of the PL-JC was matched to BC-JC using BC flavouring, Allura Red, Raspberry, and Blue colour solutions (Sensient Technologies) (Table 5). A sucrose solution (65° Brix) was used in place of the BC juice concentrate for PL-JC.

TABLE 5
Constituents of the blackcurrant and placebo juice concentrate
formulations presented as single-serve quantities.

| | Blackcurrant | | Placebo | |
|---|---|---|---|---|
| Constituents | concentrate | | concentrate | |
| Blackcurrant concentrate (65° Brix) | 26.4 | g | N/A | |
| Apple concentrate | 9.6 | g | 9.6 | g |
| Sucrose solution (65° Brix) | N/A | | 26.4 | g |
| Citric acid anhydrous | 0.12 | g | 2.4 | g |
| Clove flavour | 0.37 | g | 0.37 | g |

| | | | |
|---|---|---|---|
| Blackcurrant flavour | N/A | 0.28 | g |
| Allura Red colour solution | N/A | 0.76 | g |
| Raspberry colour solution | N/A | 0.66 | g |
| Blue colour solution | N/A | 0.05 | g |

N/A = not applicable.

### Microbial Pathogen Testing

Received powders and formulated juice concentrates were sent to AsureQuality Limited for microbial pathogen testing.

### Beverage Preparation

An independent researcher prepared the trial beverages. The required amber bottles of stored formulations were moved to 4° C. the night before a trial day. The powder drinks were prepared by blending the powders and 300 ml of water together in a NutriBullet®. Water was added to the juice concentrates to equal 300 mL and stirred to combine. Trial beverages were consumed by the participants within 1 h of being prepared.

### Anthocyanin Quantitation of Beverage Formulations

Anthocyanin concentrations of formulated trial juice concentrates and supplied powders were measured using a Dionex Ultimate 3000 Series UHPLC (ThermoFisher Scientific, San Jose, CA, USA) with PDA (photodiode array) detection at 520 and 530 nm. The formulated juice concentrates were diluted 1/1 with 5/95 formic acid/water v/v. Weighed quantities of the powder samples were dissolved in 5/95 formic acid/water v/v to give aqueous solutions of concentration between 20 and 21 mg/mL. Detected anthocyanins were quantitated using a pure standard of cyanidin 3-O-glucoside and all the results for individual and total anthocyanins are expressed as cyanidin 3-O-glucoside equivalents.

### Clinical Trial

### Recruitment and Trial Registration

Thirteen healthy individuals between 26 and 39 years old, who were recruited from the wider Palmerston North community, provided informed consent to participate in this study. During recruitment, prospective participants completed a health screening questionnaire to exclude those who had a chronic disease (e.g. heart disease, cancer), known blood-borne diseases (e.g. hepatitis), a recent viral or bacterial illness, were pregnant, were taking medication that affected blood-clotting properties or mood, had a known intolerance to blackcurrants, or a known strong reaction to needles. All methods and procedures were reviewed and approved by New Zealand's Northern B Health and Disability Ethics Committee (2021 EXP 11576).

### Sample Size

Power analysis from previous blackcurrant intervention studies measuring peripheral MAO-B activity was used to calculate the number of volunteers for this study. Our analysis revealed that 3 participants per intervention group would be required to detect a difference of 20% in MAO-B inhibition with 80% power. To account for potential participant dropout, at least six participants were recruited per format group.

### Study Design

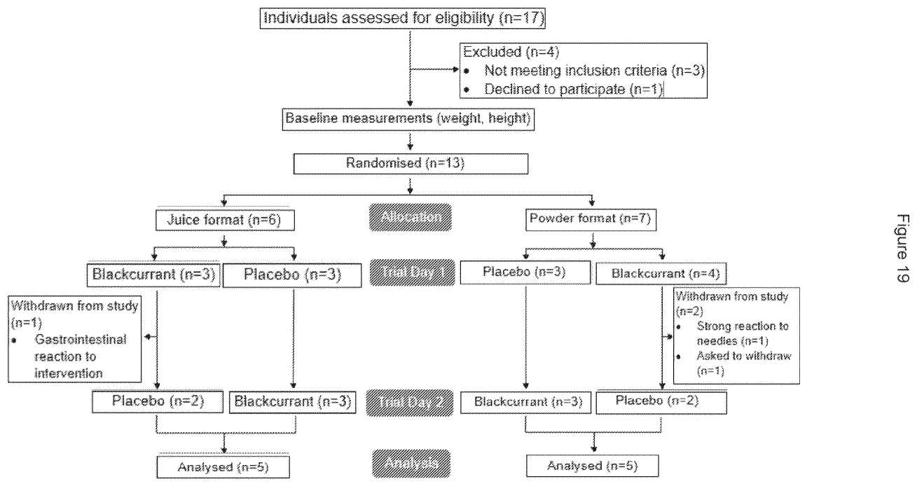

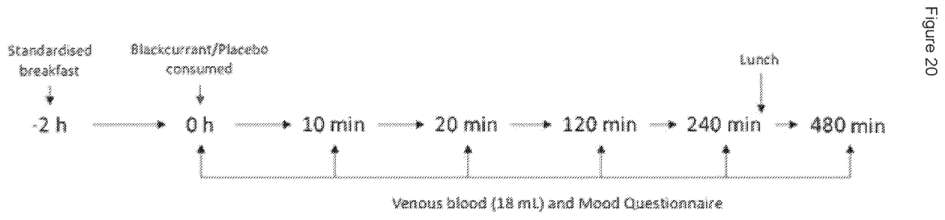

The study followed randomised, double-blind, two-arm, placebo-controlled, cross-over human intervention design. A flow chart of the study protocol is shown in FIG. 19 and the trial day procedures in FIG. 20. The trial participants were to complete two trial days, where they would consume one of the two intervention drinks (blackcurrant or placebo), with at least one-week washout between them.

Subjective feeling questionnaires and venous blood samples were collected at various timepoints over 8 h. The participants were randomly allocated (ratio 1:1) into one of the two format groups: juice concentrate, or powder, and then randomly allocated (ratio 1:1) for the order in which they received the intervention beverages. Participants and trial coordinators were masked as to which intervention drink participants consumed. Before each trial day, participants excluded foods and supplements high in polyphenol compounds from their diet, for 24 h. They fasted for at least 10 h before consuming an Almond with Vanilla One Square Meal Bar® (Cookie Time Ltd) for breakfast, 2 h before the start of the trial. Participants arrived at the PFR Clinical Facility, where they were made comfortable. After 5 min at rest, they completed visual analogue scale (VAS) subjective feelings questionnaires and then donated a venous blood sample. After which, they were served their intervention beverage and were instructed to consume it as quickly as possible. VAS mood questionnaires were completed, and venous blood samples were collected at 10, 20, 120, 240 and 480 min after finishing the beverages.

Besides the standardised breakfast and intervention beverage, participants consumed only water throughout the 480 min and a small polyphenol-low lunch (white bread sandwich with butter, mayonnaise, and poached chicken or mashed boiled egg). Participants were allowed to leave the clinical facility after the 20-min timepoint and return for subsequent timepoints if they wished.

### Subjective Mood Questionnaires

Bond-Lader Visual Analogue Mood Scales Questionnaire allows self-evaluation of mood. In total, 16 dimensions of mood are given: Alert-Drowsy, Calm-Excited, Strong-Feeble, Muzzy-Clear headed, Well Coordinated-Clumsy, Lethargic-Energetic, Contented-Discontented, Troubled-Tranquil, Mentally Slow-Quick Witted, Tense-Relaxed, Attentive-Dreamy, Incompetent-Proficient, Happy-Sad, Antagonistic-Friendly, Interested-Bored, Withdrawn-Social. The participants were required to mark on a 100-mm line to what extent each described state was appropriate to them at that moment in time. The individual responses from the 16 mood scales were combined to make three affective dimensions of alertness, contentment, and calmness (Bond and Lader 1974). Visual Analogue Mood Scales for stress, anxiety and mental fatigue on a 100-mm line anchored with “not at all” on one side of the scale and “extremely” on the other, were used to measure participants' current feelings of stress, anxiety and mental fatigue.

### Biochemical Analysis

Venous blood was collected from each participant prior to beverage consumption (0 min), and 10, 20, 120, 240 and 480 min post beverage consumption into 1×10-mL EDTA vacutainer for platelet MAO-B activity and blood glucose measurement, and 1×10-mL lithium-heparin tube for plasma anthocyanin quantification.

### Platelet MAO-B Activity

Platelets were isolated and prepared using previously described methods (Watson et al. 2015). In brief, platelets were isolated from whole blood (10 mL in 10% disodium EDTA solution), by centrifugation at 600 g at 22° C. for 3 min without a break. Platelets were stored at −80° C. until required. The protein concentration of the platelet samples was determined using the BCA Protein Assay Kit (23225, Pierce™). MAO-B activity was measured using the Amplex® Red Monoamine Oxidase Assay Kit (A12214, Invitrogen), as per the manufacturer's instructions.

### Anthocyanin & Sarmentosin Analysis

Plasma was separated from whole blood collected in lithium heparin tubes from participants by centrifugation at 4000×g for 10 min at 4° C. Plasma (1 mL) was spiked with 30 μL of 50% formic acid and 100 μL of 10 mmoL ascorbic acid then stored at −80° C. prior to shipping on dry ice to the Physiological Chemistry laboratory.

The plasma samples (350 μL) were spiked with malvidin 3-O-galactoside and further acidified with phosphoric acid prior to clean up on a SOLAμ™ Solid Phase Extraction plate. Following washing with water and acetic acid, the retained anthocyanins were eluted with methanol: formic acid (95:5), evaporated to dryness then reconstituted in acetonitrile: formic acid: water (5:3:92) prior to analysis by Liquid Chromatography-Mass Spectrometry (LC-MS). Quantifications of individual and total anthocyanins were performed using the internal standard ratio method using MultiQuant software, and all results are reported as cyanidin 3-O-glucoside equivalents. Quantification of sarmentosin was performed using an external reference standard supplied by Stephen Bloor (Callaghan Institute).

### Neurotransmitter Analysis

Overall, 111 plasma samples were analysed and the total number of samples available for each intervention type/intervention format/time point ranged between n=3 to n=7. The methodology for neurotransmitter analysis utilises a MS-probe and stable isotope coding LCMS method developed in-house at Plant & Food Research, optimised for plasma samples (Parkar et al. 2020; Watson et al. 2020). The neurotransmitter methodology includes extensive coverage of inhibitory and excitatory neurotransmitters from the tyrosine, tryptophan and glutamate metabolic pathways involved in the gut-brain axis. Briefly, metabolites measured in the tyrosine metabolic pathway were phenylethylamine (PEA), tyrosine (TYR), 3,4-dihydroxyphenylalanine (L-DOPA), dopamine (DA), 3-methoxytyramine (3-MT), 3,4-dihydroxyphenylacetic acid (DOPAC), homovanillic acid (HVA), norepinephrine (NE), 3,4-dihydroxyphenylglycol (DHPG), 3-methoxy-4-hydroxyphenylglycol (MHPG), normetanephrine (NM), epinephrine (E), metanephrine (MN), and vanillylmandelic acid (VMA). In the tryptophan metabolic pathway were tryptophan (TRP), kynurenine (KYN), kynurenic acid (KA), xanthurenic acid (XA), quinolinic acid (QA), 5-hydroxytryptophan (5-HTP), serotonin (5-HT), 5-hydroxyindoleacetic acid (5-HIAA) and melatonin (MT). Further, for glutamate metabolism the metabolites measured were glutamic acid (GLU), alpha-aminobutyric acid (AABA) and gamma-aminobutyric acid (GABA).

Other endogenous analytes associated with the gut brain axis measured were glycine (GLN), serine (SER), histamine (HIS), adenosine (ADO) and cortisol (CORT). Samples were derivatised in three stages to acetylate alcohol and amine functional groups and alkylate carboxylic acid groups prior to LC-MS analysis. A multiple derivatisation strategy was found to be necessary to acetylate the less reactive alkyl hydroxyl and carboxylic acid groups. Deuterated internal standards (IS) for analytes were added at the beginning of sample work-up to correct for recovery.

To facilitate quantitation and to correct for matrix effects during analysis, a labelled IS for each analyte was prepared (IS-XdP) by derivatising a mixed neurotransmitter standard, as described for the samples, with the exception that deuterated acetic anhydride [d6] and deuterated trifluoroethanol [d3] were used in place of unlabelled acetic anhydride and unlabelled trifluoroethanol. The IS-XdP standard was added to the samples after sample work-up.

Briefly, acetic anhydride, sodium bicarbonate/carbonate buffer, acetonitrile and the mixed IS comprising of deuterated analytes was added to each plasma sample (100 μL). Samples were evaporated to dryness overnight then derivatised with acetic anhydride and trifluoroethanol at 70° C. for 4 h and the temperature reduced to 50° C. before being left to react overnight. Samples were once again taken to dryness and further derivatised with acetic anhydride before the addition of water, acetonitrile and the IS-XdP. Samples were filtered through a 96-well glass fibre plate and collected in a 96-deepwell polypropylene plate prior to analysis by LCMS. LCMS experiments were carried out on a 7500 QTrap triple quadrupole/linear ion trap (QqLIT) mass spectrometer equipped with a Turbo V™ ion source and electrospray ionisation (ESI) probe (AB Sciex, Concord, ON, Canada) coupled to a Shimadzu Nexera LC40 UHPLC (Shimadzu, Tokyo, Japan). MS data was acquired in the positive mode using a multiple reaction monitoring (MRM) method.

### Blood Glucose

Blood glucose concentrations of whole blood was measured using a HemoCue® 201 DM System (HemoCue®, Ängelholm, Sweden) blood glucose analyser.

### Statistics

Chemistry data presented in this report are means±standard errors of the mean. Monoamine oxidase-B activity, subjective measures and glucose data are presented as means±standard errors of the mean. Comparisons of means between timepoints, treatments and formats were made using Analysis of Variance (ANOVA) from a linear mixed effects model, with fixed effects for format, study day, treatment, time, and their interactions, and random effects for participant, participant×study day, participant×treatment and participant×time-point. The models were fitted with R package ImerTest, and least significant differences (LSDs) for comparing means were calculated post hoc using the R package predictmeans. Statistical significance for all indices was set at p<0.05 with a confidence level of 95%.

### Neurotransmitter Data Analysis

R version 4.2.1 was used for analysis and data visualisation of neurotransmitter results. All observations were standardised as the difference to the first measurement of the time-course (0 min). Spearman's rank-sum correlation matrices of standardised neurotransmitter concentrations and MAO-B inhibition were computed for each condition using the package ‘PerformanceAnalytics’. One-dimensional plots of variables showing significant correlations with MAO-B inhibition in Intervention formats and their respective correlation coefficients in placebo formats were visualised using the R base function. A multivariate approach was employed using standardised, median-centred and scaled neurotransmitter concentrations as variables. Using the ‘factoMineR’ package, principal component analysis was performed and variable loadings with strongest contributions to components 1 and 2 were visualised as correlation circle plots.

For univariate statistical analyses of standardised neurotransmitters, linear mixed effects models were computed using R package ‘ImerTest’, to allow for both, fixed and random effects of multiple factors. The model fitted had fixed effect factors for formulation (powder vs juice), treatment (blackcurrant vs placebo) and time; the random effects were participant, participant×treatment and participant×time.

Each participant only received one formulation, so there was no need for a participant×formulation random effect. Where ANOVA indicated there were significant (p<0.01) treatment effects or interactions, predicted means were obtained and pairwise comparisons computed on Fisher's Least Significant Difference (LSD, α=0.05) conducted using the ‘predictmeans’ package.

### Results

### Anthocyanin Content of Juice and Powders

The blackcurrant anthocyanin contents of the formulated trial juice concentrates and powders before water was added (to make 300-mL beverages), are shown in Table 6. Based on the total anthocyanin results, the blackcurrant juice concentrate contained 2179.4 μg/g wet weight total anthocyanins, and the blackcurrant powder contained 33720.4 μg/g dry weight total anthocyanins. The placebo juice concentrate and powder formulation contained no detectable blackcurrant anthocyanins.

TABLE 6
Blackcurrant anthocyanin content in the blackcurrant and placebo juice concentrates,
and blackcurrant and placebo powders. Juice concentrates are reported in μg/g
wet weight, and powders reported in μg/g dry weight as received.

| | Blackcurrant | Blackcurrant | Placebo | Placebo |
|---|---|---|---|---|
| Compound | concentrate | powder | concentrate | powder |
| Delphinidin 3-O- | 169.9 | 3677.5 | nd | nd |
| glucoside | | | | |
| Delphinidin 3-O- | 840.4 | 13272.0 | nd | nd |
| rutinoside | | | | |
| Cyanidin 3-O-glucoside | 104.7 | 1507.9 | nd | nd |
| Cyanidin 3-O-rutinoside | 1032.1 | 14920.1 | nd | nd |
| Petunidin 3-O-rutinoside | 15.1 | 76.8 | nd | nd |
| Pelargonidin 3-O- | 6.6 | 109.4 | nd | nd |
| rutinoside | | | | |
| Peonidin 3-O-rutinoside | 10.6 | 156.7 | nd | nd |
| TOTAL | 2179.4 | 33720.4 | nd | nd |

nd: not detected.

### Trial Participants

Ten participants completed both trial days out of the 13 who were recruited (FIG. 19).

Data of the three participants who did not complete both trial days were not included in the analyses for this report. The anthropometric data of the ten participants are shown in Table 7. Of these ten, five were allocated to the juice concentrate format, and five were allocated to the powder format. Despite withdrawals by some participants, the number that completed both trial days were within what was required to detect statistically significant MAO-B enzyme inhibition from consuming the blackcurrant formats.

### Participant Withdrawals and Reactions

TABLE 7
Anthropometric characteristics of participants of the two format
groups: juice concentrate and powder. Data are means ± SEM,
and minimum and maximum values from n = 5 individuals per group.

| | | | Powder | |
|---|---|---|---|---|
| | Juice concentrate format | | format | |
| Variable | Average | Range | Average | Range |
| Age (years) | 33 ± 1.7 | 29-39 | 30 ± 2.1 | 26-39 |
| Height (cm) | 174 ± 4.0 | 164-187 | 174 ± 4.0 | 159-185 |
| Weight (kg) | 83 ± 8.8 | 59-119 | 75 ± 3.8 | 63-87 |
| Body Mass Index | 28 ± 3.8 | 22-45 | 25 ± 0.4 | 24-26 |
| (BMI) | | | | |

Of the three participants who did not complete both trial days, one asked to withdraw after completing the first day; one withdrew after having a strong reaction to needles; and the other participant withdrew after having a temporary adverse gastrointestinal reaction to the intervention drink.

### Total Anthocyanin Dose

The absolute anthocyanin doses consumed by participants in the two Neuroberry® blackcurrant interventions were not equivalent (Table 8) owing to the different dose standardisations used for each format. Participants in the BC-JC group all consumed 300 mL of single-strength blackcurrant juice, whereas participants in the BC-P group consumed a standardised dose of anthocyanins (7.8 mg total anthocyanins/kg bodyweight). Consequently, participants in the PC-P group consumed approximately 7.8 times more anthocyanins per kg bodyweight than volunteers in the BC-JC group.

TABLE 8
Average consumption of blackcurrant formulations and total anthocyanins per
format group. Data are means ± SEM from n = 5 individuals per format group.

| | Blackcurrant juice | Blackcurrant |
|---|---|---|
| Variable | concentrate | powder |
| Weight of intervention consumed* (g) | 36.2 ± 0.0 | 17.0 ± 0.9 |
| Total anthocyanins (mg) | 78.8 ± 0.0 | 590.8 ± 32.1 |
| Total anthocyanins per kg bodyweight (mg) | 1.0 ± 0.1 | 7.8 ± 0.0 |

*Without additional water.

### Anthocyanin and Sarmentosin Bioavailability

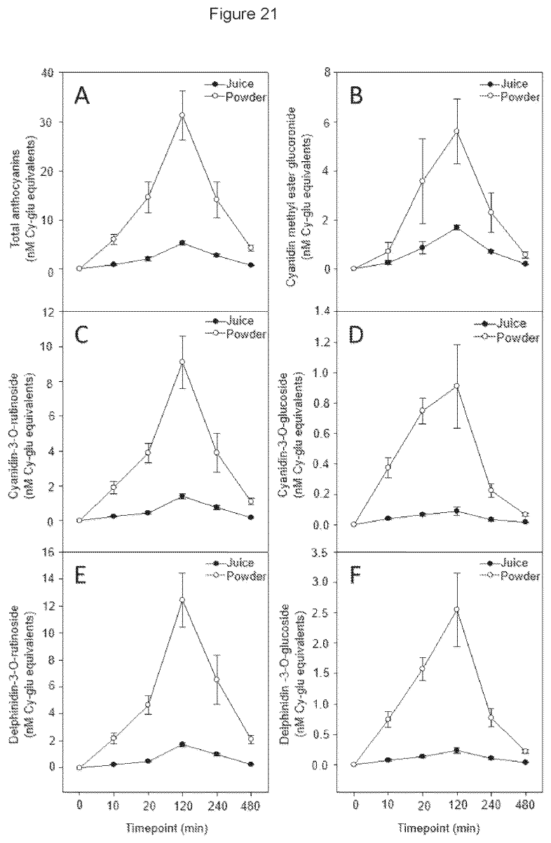

The concentrations of total anthocyanins, cyanidin methyl ester glucuronide, cyanidin 3-O-runtinoside, cyanidin 3-O-glucoside, delphinidin 3-O-runtinoside, and delphinidin 3-O-glucosides measured in plasma following the consumption of the blackcurrant interventions were measured (FIG. 21). No blackcurrant anthocyanins were detectable in the plasma of the placebo (PL) interventions (data not shown). The concentrations of the anthocyanins were higher in the BC-P plasma than in the BC-JC for all timepoints measured post-consumption. Maximum total anthocyanin concentrations in plasma were 25.7±4.1 nM and 3.6±0.4 nM for BC-P and BC-JC, respectively. The peak concentrations of all anthocyanins were measured at 120 min for both formats.

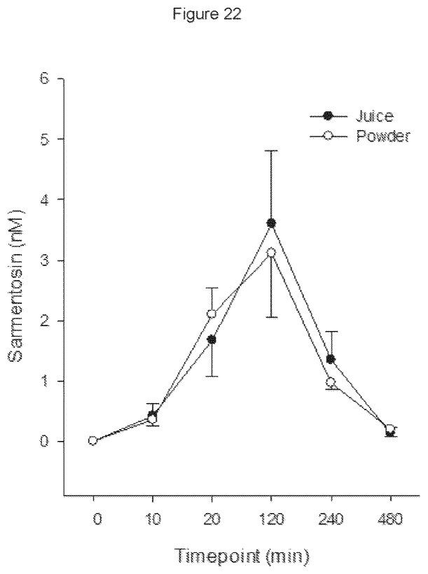

Sarmentosin bioavailability in plasma following the consumption of the BC beverages is shown in FIG. 22. Similarly, to anthocyanin bioavailability, peak concentrations of sarmentosin were measured at 120 min. The bioavailability profiles of sarmentosin are similar in both formats. It should be noted however that the analysis methodology was not optimised for sarmentosin, and that recovery of sarmentosin through the clean-up protocol was poor. Therefore, while relative concentrations of sarmentosin measured for the two BC formats can be used to inform bioavailability profiles, the absolute sarmentosin concentrations is likely to be under-reported. Any further analyses should employ a stepwise clean-up protocol for anthocyanins and sarmentosin, and separate LC-MS analysis.

### MAO-B Inhibition and Blood Glucose

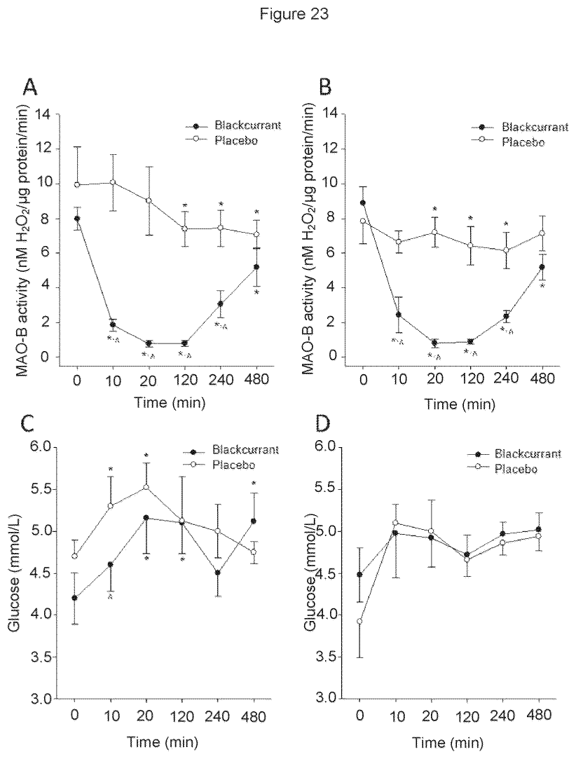

Blood glucose concentrations and platelet MAO-B enzyme activities after consuming the Neuroberry® blackcurrant and placebo interventions are shown in FIG. 23. ANOVA using a mixed effects model (Table 9), revealed that format (i.e., powder or juice) did not have a significant effect on MAO-B enzyme activity (p=0.561). However, significant treatment (BC vs placebo) (p=0.011), time (p<0.001), and treatment×time (p<0.001) interactions were detected.

TABLE 9
Analysis of variance (ANOVA) results from mixed effects models
to determine the effects of format, treatment, time, plus treatment
× time interactions on MAO-B enzyme activity and blood
glucose after consuming trial beverages. Data are F values and
p values, with p < 0.05 indicating a significant effect.

| | |
|---|---|
| MAO-B activity | Blood glucose |

| Interactions | F | p | F | p |
|---|---|---|---|---|
| Format | 0.4 | 0.561 | 0.1 | 0.606 |
| Treatment | 24.8 | 0.011 | 0.7 | 0.747 |
| Time | 28.6 | <0.001 | 3.7 | 0.043 |
| Treatment × Time | 112.6 | <0.001 | 1.8 | 0.136 |

MAO-B activity reduced following the consumption of all trial beverages, but there was a significant reduction in MAO-B activity from baseline (0 min), 10 min after the consumption of the blackcurrant beverages (−76.1%±7.9 and −75.9%±9.7; BC-JC, BC-P, respectively). Maximum MAO-B inhibition was measured for both formats at 20 min post-consumption (−89.6%±2.2, −90.5%±2.8, BC-JC, BC-P, respectively) and maintained at this level up to 120 min. Significant inhibition of MAO-B persisted for the duration of the study so that enzyme activity was significantly lower in both blackcurrant formats 480 min after consumption (−35.3%±11.9 and −38.3%±11.7; BC-JC, BC-P, respectively).

Small, but significant reductions in MAO-B activity from baseline were measured following the consumption of both placebo formulations with maximum inhibition of these beverages at −8.6%±6.2 and −21.0±6.3 for PL-JC and PL-P at 480 min and 240 min, respectively.

Statistical analysis (Table 9) revealed a significant time effect on blood glucose concentrations (p=0.043). On average, blood glucose concentrations typically rose after consuming the trial beverages (10 and 20 min) and remained above baseline concentrations during the remaining timepoints. No significant changes in blood glucose were observed following the consumption of the powder beverages (BC-P and PL-P). In comparison, a significant increase in blood glucose from baseline (0-min) concentrations were measured after consuming PL-JC (10 min and 20 min) and BC-JC (20 min, 120 min and 480 min).

### VAS Subjective Mood Scores

### Bond-Lader Questionnaire

TABLE 10
Analysis of variance (ANOVA) results from mixed effects models to determine the effects of format,
treatment, time, and their interactions on descriptive items pertaining to alertness after consuming
trial beverages. Data are F values and p values with p < 0.05 indicating a significant effect.

| | | | | | | | | | | | Mentally | | | | | | | |
|---|---|---|---|---|---|---|---|---|---|---|---|---|---|---|---|---|---|---|
| | Alert/ | | Strong/ | | Muzzy/ | | Well-coordinated/ | | Lethargic/ | | slow/Quick- | | Attentive/ | | Incompetent/ | | Interested/ | |
| | Drowsy | | Feeble | | Clear-headed | | Clumsy | | Energetic | | witted | | Dreamy | | Proficient | | Bored | |
| Interactions | F | p | F | p | F | p | F | p | F | p | F | p | F | p | F | p | F | p |
| Format | 0.6 | 0.458 | 0.1 | 0.777 | 0.1 | 0.734 | 0.6 | 0.453 | 0.5 | 0.510 | 0.2 | 0.657 | 1.9 | 0.220 | 0.1 | 0.738 | 1.6 | 0.257 |
| Treatment | 0.0 | 0.939 | 0.2 | 0.713 | 0.8 | 0.411 | 0.1 | 0.725 | 0.8 | 0.406 | 0.8 | 0.405 | 0.0 | 0.933 | 0.2 | 0.695 | 0.1 | 0.744 |
| Time | 1.7 | 0.229 | 1.3 | 0.340 | 0.8 | 0.414 | 1.6 | 0.260 | 3.9 | 0.036 | 1.8 | 0.205 | 1.5 | 0.297 | 2.2 | 0.131 | 3.2 | 0.076 |
| Treatment × Time | 2.1 | 0.076 | 0.8 | 0.572 | 0.7 | 0.601 | 1.9 | 0.116 | 1.7 | 0.144 | 0.8 | 0.521 | 0.7 | 0.597 | 1.2 | 0.299 | 1.3 | 0.283 |
| Format × Treatment × | 1.6 | 0185 | 1.3 | 0.288 | 1.5 | 0.222 | 1.4 | 0.247 | 0.9 | 0.461 | 0.7 | 0.599 | 1.8 | 0.137 | 2.1 | 0.075 | 2.9 | 0.024 |
| Time | | | | | | | | | | | | | | | | | | |

TABLE 11
Analysis of variance (ANOVA) results from mixed effects models to determine the effects of format, treatment, time,
and their interactions on descriptive items pertaining to contentment and calmness (Tense/Excited and Tense/Relaxed)
after consuming trial beverages. Data are F values and p values with p < 0.05 indicating a significant effect.

| | | | Troubled/ | | | | Unfriendly/ | | Withdrawn/ | | | | | |
|---|---|---|---|---|---|---|---|---|---|---|---|---|---|---|
| Tim point | Contented/ Discontented | | Tranquil | | Happy / Sad | | Friendly | | Sociable | | Calm/Excited | | Tense/Relaxed | |
| (min) | F | p | F | p | F | p | F | p | F | p | F | p | F | p |
| Format | 0.1 | 0.717 | 0.0 | 0.848 | 0.7 | 0.423 | 0.2 | 0.667 | 0.1 | 0.793 | 0.1 | 0.787 | 0.1 | 0.807 |
| Treatment | 0.0 | 0.914 | 0.0 | 0.860 | 0.9 | 0.402 | 0.0 | 0.851 | 0.4 | 0.543 | 0.3 | 0.614 | 0.1 | 0.761 |
| Time | 1.3 | 0.327 | 0.6 | 0.732 | 0.4 | 0.847 | 1.9 | 0.178 | 2.9 | 0.083 | 0.5 | 0.791 | 1.1 | 0.401 |
| Treatment × Time | 0.8 | 0.581 | 1.0 | 0.456 | 1.1 | 0.373 | 9.8 | 0.000 | 1.3 | 0.283 | 0.6 | 0.690 | 1.5 | 0.195 |
| Format × Treatment × | 1.6 | 0.172 | 0.2 | 0.960 | 2.8 | 0.028 | 12.0 | 0.000 | 1.9 | 0.111 | 2.5 | 0.045 | 0.3 | 0.926 |
| Time | | | | | | | | | | | | | | |

For descriptors related to alertness, a significant time interaction was observed for Lethargy/Energetic scores and a significant format×treatment×time interaction was measured for Interested/Bored scores. No significant effects were observed of format, time, treatment their interactions were observed for the rest of alertness-related items.

A significant treatment×time interaction was measured observed for Unfriendly/Friendly scores. Significant format×treatment×time interactions was also measured for Happy/Sad and Unfriendly/Friends, items related to contentment. A significant format×treatment×time interaction was also observed for Calm/Excited scores, a descriptor relating to contentment.

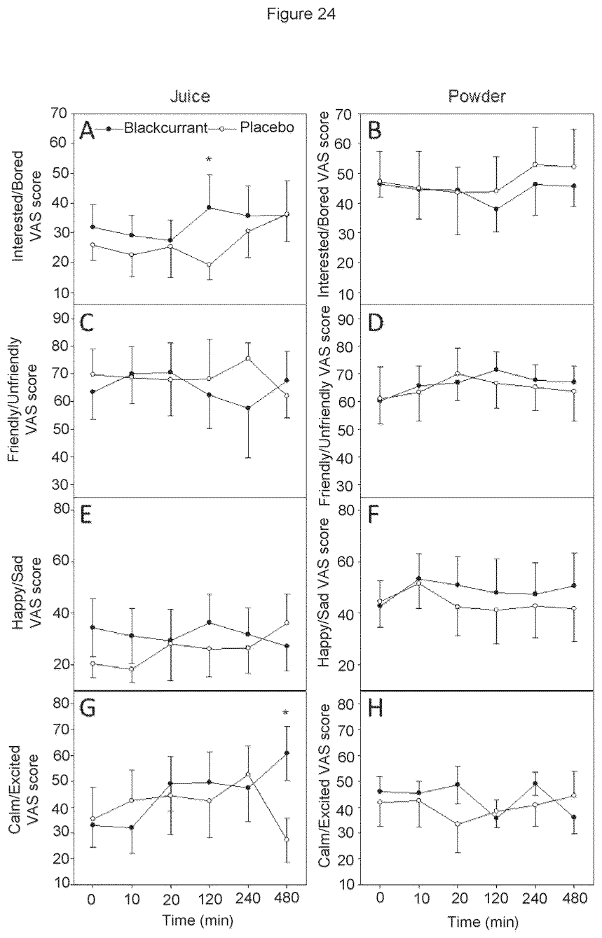

Descriptive items from the Bond-Lader Questionnaire found to have significant format×treatment×time interactions are plotted in FIG. 24. Significantly (p<0.05) higher Interested/Bored and Calm/Excited scores were observed in the BC-JC compared with the PL-JC intervention at 120 min and 480 min after, respectively. No significant differences between BC-P and PL-P for these items were observed at the same timepoints.

TABLE 12
Analysis of variance (ANOVA) results from mixed effects models
to determine the effects of format, treatment, time plus
their interactions on alertness, calmness and contentment
after consuming trial beverages. Data are F values and p
values with p < 0.05 indicating a significant effect.

| | Alertness | | Calmness | | Contentment | |
|---|---|---|---|---|---|---|
| Interactions | F | p | F | p | F | p |
| Format | 0.3 | 0.606 | 0.0 | 0.935 | 0.1 | 0.828 |
| Treatment | 0.1 | 0.747 | 0.0 | 0.921 | 0.0 | 0.871 |
| Time | 3.9 | 0.043 | 0.4 | 0.832 | 1.7 | 0.209 |
| Treatment × Time | 1.6 | 0.174 | 0.9 | 0.481 | 0.9 | 0.508 |
| Format × Treatment × Time | 1.3 | 0.262 | 1.9 | 0.108 | 1.8 | 0.131 |

When the descriptive items from the Bond-Lader Questionnaire were categorised to give composite scores for alertness, calmness and contentment, a significant time effect was observed for alertness, but not for calmness and contentment. No significant format, treatment, treatment×time and format×treatment×time interactions were detected for these categories.

TABLE 13
Alertness, calmness and contentment scores (from Bond-Lader Questionnaire) after consuming
blackcurrant juice or powder and their corresponding placebos. Values are means ± SEM.

| Timepoint | Alertness | | Calmness | | Contentment | |
|---|---|---|---|---|---|---|
| (min) | Placebo | Blackcurrant | Placebo | Blackcurrant | Placebo | Blackcurrant |
| Juice | | | | | | |
| 0 | 64.6 ± 3.6 | 62.6 ± 7.2 | 53.2 ± 8.0 | 62.9 ± 8.7 | 63.7 ± 7.8 | 61.0 ± 11.0 |
| 10 | 71.2 ± 2.4 | 66.1 ± 5.9 | 59.4 ± 11.7 | 65.7 ± 9.5 | 70.0 ± 10.0* | 67.2 ± 10.6 |
| 20 | 72.6 ± 4.5* | 66.1 ± 6.7 | 59.4 ± 14.2 | 58.4 ± 7.6 | 68.6 ± 12.6 | 69.6 ± 11.0* |
| 120 | 70.3 ± 2.5 | 65.1 ± 6.9 | 60.5 ± 13.2 | 58.5 ± 9.1 | 68.8 ± 13.2 | 63.0 ± 11.9 |
| 240 | 61.9 ± 4.2 | 61.1 ± 7.8 | 52.3 ± 10.5 | 55.5 ± 8.0 | 66.7 ± 8.5 | 61.7 ± 11.6 |
| 480 | 54.0 ± 5.4* | 61.1 ± 7.7 | 63.3 ± 11.0* | 49.0 ± 5.4*\hat{ } | 61.3 ± 12.3 | 67.6 ± 9.6* |
| Powder | | | | | | |
| 0 | 52.3 ± 9.0 | 53.4 ± 5.8 | 51.3 ± 4.8 | 52.5 ± 6.0 | 56.6 ± 13.8 | 55.9 ± 9.0 |
| 10 | 58.2 ± 11.2 | 61.8 ± 6.9* | 53.2 ± 7.6 | 50.3 ± 6.4 | 57.4 ± 12.8 | 56.8 ± 8.8 |
| 20 | 61.5 ± 12.5* | 62.8 ± 6.5* | 60.9 ± 7.9* | 49.5 ± 6.2? | 61.2 ± 13.7 | 57.2 ± 9.0 |
| 120 | 57.0 ± 10.0 | 67.8 ± 7.4*? | 59.0 ± 5.0 | 58.4 ± 6.6 | 61.7 ± 11.3 | 58.6 ± 9.4 |
| 240 | 52.1 ± 10.9 | 59.2 ± 7.5 | 55.6 ± 3.4 | 52.2 ± 4.1 | 55.9 ± 12.3 | 57.2 ± 9.0 |
| 480 | 49.7 ± 9.4 | 55.7 ± 7.2 | 54.4 ± 6.8 | 57.4 ± 5.8 | 57.6 ± 13.5 | 55.2 ± 10.0 |

*Denotes significant difference from 0 min (p < 0.05).
\hat{ }Denotes significant difference from placebo at corresponding time point (p < 0.05).

Participants' alertness scores significantly increased 20 min after consuming the PL-JC intervention, then declined thereafter, so that alertness scores at 480 min were significantly lower than baseline (0 min) scores (Table 11). Alertness scores did not significantly change from baseline (0 min) after BC-JC consumption at any timepoints. For the powder formats, alertness scores were significantly higher than baseline (0 min) scores 20 min after consuming the PL-P intervention. A significant increase in alertness was also observed 10 min, 20 min and 120 min after BC-P consumption. Additionally, BC-P alertness scores 120 min post-consumption were significantly higher than placebo (PL-P) scores at this timepoint.

Mean calmness was significantly higher 480 min after consuming the juice placebo beverage (PL-JC), while significantly lower calmness was measured at this timepoint after drinking the blackcurrant juice (BC-JC), so that the calmness scores between treatment groups at this timepoint were significantly different (p<0.05) (Table 11). A significant increase in calmness was measured 20 min after PL-P consumption, while no change in calmness was measured in the BC-P intervention group. Calmness scores in the PL-P were significantly higher 20 min after consumption than in the BC-P intervention at this timepoint.

An immediate increase in contentment was measured in the PL-JC intervention group from baseline (0 min) scores (Table 11). This trend was also observed in the BC-JC group, with significantly higher contentment scores at 20 min and 480 min after beverage consumption. For the powder formats, neither beverage (PL-P or BC-P) resulted in significant changes in contentment scores at any of the timepoints measured.

### Stress, Anxiety and Mental Fatigue

TABLE 14
Analysis of variance (ANOVA) results from mixed effects models
to determine the effects of format, treatment, time plus
their interactions on stress, anxiety and mental fatigue
after consuming trial beverages. Data are F values and p
values with p < 0.05 indicating a significant effect.

| | Stress | | Anxiety | | Mental fatigue | |
|---|---|---|---|---|---|---|
| Interaction | F | p | F | p | F | p |
| Format | 0.0 | 0.891 | 0.0 | 0.842 | 0.0 | 0.971 |
| Treatment | 1.9 | 0.263 | 0.9 | 0.378 | 0.1 | 0.727 |
| Time | 2.1 | 0.161 | 0.5 | 0.794 | 0.4 | 0.842 |
| Treatment × Time | 3.0 | 0.021 | 1.8 | 0.134 | 3.3 | 0.013 |
| Format × Treatment × Time | 2.4 | 0.051 | 0.8 | 0.537 | 0.4 | 0.865 |

No significant format, treatment or time effects were observed for subjective measures of stress, anxiety, or mental fatigue (Table 14). There were, however, significant treatment x time effects for stress (p=0.021), and mental fatigue (p=0.013). A near-significant format×treatment×time interaction was detected for stress (p=0.051), but not for anxiety and mental fatigue.

The placebo interventions of both formats tested in this study (PL-JC and PL-P) had no significant effects on subjective stress scores. A significant decline in stress scores was observed after consuming BC-JC (20 min and 240 min) and BC-P (20 min, 120 min and 480 min). No significant treatment differences in stress scores were observed between PL-JC and BC-JC at any of the measurement timepoints. In comparison, stress scores measured at baseline (0 min), 10 min, 20 min and 240 min were significantly higher in the BC-P group than in the PL-P group.

No significant change in anxiety scores were observed after consuming either of the powder interventions (PL-P and BC-P). Anxiety scores, however, were significantly lower than baseline (0 min) scores at 10 min and 480 min after PL-JC and BC-JC consumption. Significantly higher anxiety scores were measured at baseline (0 min), 10 min and 20 min in the BC-P group than with the corresponding placebo intervention (PL-P) at these time points.

No significant change in scores for mental fatigue were observed after consuming BC-JC, while a significant increase in this parameter was observed 240 min and 480 min after consuming the placebo intervention (PL-JC). Inversely, consuming the placebo powder (PL-P) had no significant effect on mental fatigue scores, while significant reductions from baseline (0 min) scores were measured 20 min, 120 min and 240 min after consuming the BC-P intervention. In addition, mental fatigue scores at baseline (0 min) and 480 min after BC-P consumption were significantly higher than scores after.

TABLE 15
Stress, anxiety and mental fatigue responses (on 100-mm visual analogue scales (VAS), anchored
with “not at all” and “extremely” on either end) after consuming blackcurrant
juice or powder and their corresponding placebos. Values are means ± SEM for each parameter.

| | Stress | | Anxiety | | Mental fatigue | |
|---|---|---|---|---|---|---|
| Time (min) | Placebo | Blackcurrant | Placebo | Blackcurrant | Placebo | Blackcurrant |
| Juice | | | | | | |
| 0 | 30.1 ± 12.5 | 33.8 ± 10.2 | 32.7 ± 12.0 | 36.5 ± 10.9 | 22.3 ± 6.6 | 32.2 ± 11.3 |
| 10 | 23.9 ± 14.0 | 31.3 ± 11.5 | 23.5 ± 15.5* | 29.9 ± 12.8 | 32.3 ± 13.0 | 27.4 ± 6.6 |
| 20 | 23.3 ± 14.8 | 24.5 ± 8.8* | 26.7 ± 15.7 | 32.0 ± 14.3 | 32.6 ± 13.2 | 30.4 ± 7.7 |
| 120 | 24.8 ± 14.3 | 32.1 ± 13.5 | 28.5 ± 15.9 | 37.0 ± 16.1 | 34.1 ± 14.2 | 28.0 ± 7.3 |
| 240 | 33.6 ± 16.8 | 23.6 ± 9.3* | 29.9 ± 15.9 | 32.7 ± 13.5 | 40.7 ± 13.8* | 30.7 ± 9.2 |
| 480 | 29.5 ± 13.9 | 31.2 ± 13.9 | 27.9 ± 14.0 | 24.1 ± 9.6* | 51.1 ± 12.0* | 37.6 ± 7.6 |
| Powder | | | | | | |
| 0 | 27.0 ± 16.1 | 52.5 ± 10.8? | 30.2 ± 13.7 | 48.9 ± 13.4? | 30.5 ± 16.6 | 58.6 ± 13.1? |
| 10 | 31.1 ± 16.8 | 51.4 ± 11.9? | 37 ± 16.7 | 55.0 ± 12.3? | 38.3 ± 18.5 | 45.3 ± 17.8 |
| 20 | 26.4 ± 18.0 | 44.9 ± 13.5?* | 29.5 ± 17.9 | 45.6 ± 13.7? | 28.7 ± 18.3 | 40.4 ± 16.4* |
| 120 | 27.3 ± 15.4 | 36.7 ± 15.4* | 32.3 ± 15.3 | 41.4 ± 15.2 | 35.8 ± 16.9 | 41.0 ± 17.2* |
| 240 | 32.2 ± 16.7 | 46.9 ± 14.1? | 34.1 ± 16.6 | 40.6 ± 14.4 | 40.5 ± 17.5 | 42.0 ± 16.3* |
| 480 | 27.8 ± 17.1 | 38.0 ± 14.5* | 36.2 ± 16.9 | 44.4 ± 14.3 | 29.6 ± 15.1 | 48.4 ± 48.4? |

*Denotes significant difference from 0 min (p < 0.05).
\hat{ }Denotes significant difference from placebo at corresponding time point (p < 0.05).

### Circulating Neurotransmitter Concentrations

Concentrations of 32 endogenous neurotransmitters and other compounds associated with the gut brain axis were measured in plasma for 13 participants. Of the ten participants who completed both trial days, comprising the blackcurrant intervention and placebo interventions, five participants (four receiving the juice format, and one receiving the powder format) had samples taken for each time point. Three further participants, all receiving the powder format, had one missing time point for each trial day. The remaining five participants completed only the blackcurrant intervention trial day, two participants receiving the juice format and three the powder format.

Analysis of Variance revealed significant (p<0.01) treatment×time interactions for five neurotransmitter metabolites associated with the tyrosine metabolic pathway (PHE, DOPAC, DHPG, MHPG and VMA) and 5-HIAA, a neurotransmitter from the tryptophan metabolic pathway (Table 16). Format×treatment×time interactions were also detected for PHE, NE, KA and GLU.

TABLE 16
Analysis of variance (ANOVA) results from mixed effects models to determine the
effects of treatment and time plus their interactions on circulating neurotransmitters
associated with the tyrosine, tryptophan and glutamate metabolic pathways after
consuming trial beverages. Data are F values and p values. Treatment x time
effects significant at p < 0.01 are bolded and highlighted in red.

| | | | | | | Treatment × | | Format × Treatment × | |
|---|---|---|---|---|---|---|---|---|---|
| Format | | Time | | Treatment | | Time | | Time | |
| F | p | F | p | F | p | F | p | F | p |

Tyrosine metabolites

| | | | | | | | | | | |
|---|---|---|---|---|---|---|---|---|---|---|
| PEA | 1.2 | 0.298 | 0.9 | 0.493 | 0.3 | 0.617 | 2.5 | 0.038 | 0.3 | 0.891 |
| PHE | 0.8 | 0.409 | 20.5 | 0.000 | 8.5 | 0.014 | 3.7 | 0.006 | 3.9 | 0.004 |
| TYR | 2.5 | 0.146 | 25.2 | 0.000 | 1.3 | 0.273 | 2.0 | 0.096 | 1.5 | 0.203 |
| DOPA | 1.5 | 0.243 | 0.5 | 0.739 | 0.0 | 0.904 | 0.2 | 0.978 | 0.3 | 0.907 |
| DA | 0.3 | 0.575 | 3.7 | 0.018 | 9.3 | 0.013 | 2.4 | 0.044 | 2.3 | 0.058 |
| 3-MT | 1.8 | 0.213 | 1.4 | 0.275 | 3.1 | 0.105 | 2.5 | 0.040 | 0.0 | 1.000 |
| DOPAC | 0.1 | 0.744 | 6.8 | 0.001 | 4.2 | 0.165 | 3.9 | 0.004 | 1.2 | 0.321 |
| HVA | 0.0 | 0.975 | 5.4 | 0.007 | 16.0 | 0.107 | 2.8 | 0.024 | 2.5 | 0.040 |
| NE | 0.1 | 0.764 | 1.7 | 0.211 | 1.9 | 0.205 | 0.6 | 0.694 | 4.6 | 0.002 |
| DHPG | 0.5 | 0.506 | 3.8 | 0.031 | 0.2 | 0.655 | 5.2 | 0.001 | 3.0 | 0.019 |
| MHPG | 0.4 | 0.564 | 1.8 | 0.191 | 2.2 | 0.166 | 6.7 | 0.000 | 0.5 | 0.796 |
| NM | 0.0 | 0.976 | 1.5 | 0.240 | 0.2 | 0.694 | 2.2 | 0.062 | 3.2 | 0.012 |
| MN | 0.1 | 0.825 | 0.3 | 0.908 | 1.1 | 0.312 | 2.7 | 0.029 | 1.3 | 0.295 |
| VMA | 1.3 | 0.285 | 6.8 | 0.001 | 1.8 | 0.213 | 6.7 | 0.000 | 1.9 | 0.109 |

Tryptophan metabolites

| | | | | | | | | | | |
|---|---|---|---|---|---|---|---|---|---|---|
| TRP | 0.5 | 0.516 | 9.1 | 0.000 | 0.1 | 0.780 | 0.1 | 0.986 | 0.7 | 0.616 |
| KYN | 0.5 | 0.477 | 3.2 | 0.029 | 0.1 | 0.736 | 0.2 | 0.944 | 0.5 | 0.783 |
| KA | 0.0 | 0.998 | 14.4 | 0.000 | 0.6 | 0.453 | 0.8 | 0.574 | 4.1 | 0.003 |
| XA | 0.4 | 0.542 | 11.8 | 0.000 | 3.7 | 0.088 | 0.8 | 0.524 | 2.2 | 0.072 |
| QA | 0.0 | 0.933 | 4.2 | 0.019 | 3.9 | 0.079 | 2.0 | 0.089 | 3.1 | 0.015 |
| 5-HTP | 0.1 | 0.827 | 1.5 | 0.250 | 0.9 | 0.384 | 2.3 | 0.058 | 1.7 | 0.138 |
| 5-HT | 8.4 | 0.033 | 2.7 | 0.061 | 3.4 | 0.103 | 2.4 | 0.049 | 2.0 | 0.088 |
| 5-HIAA | 4.8 | 0.052 | 11.4 | 0.000 | 11.4 | 0.011 | 5.9 | 0.000 | 2.1 | 0.079 |

Glutamate metabolites and other metabolites associated with the GBA

| | | | | | | | | | | |
|---|---|---|---|---|---|---|---|---|---|---|
| GLU | 0.2 | 0.667 | 2.4 | 0.084 | 4.0 | 0.067 | 2.2 | 0.061 | 8.6 | 0.000 |
| AABA | 2.0 | 0.184 | 1.3 | 0.330 | 0.4 | 0.560 | 2.5 | 0.037 | 2.3 | 0.057 |
| GABA | 0.1 | 0.717 | 0.5 | 0.765 | 0.6 | 0.442 | 1.9 | 0.108 | 1.3 | 0.276 |
| GLN | 0.2 | 0.681 | 4.2 | 0.025 | 3.5 | 0.089 | 2.6 | 0.039 | 2.5 | 0.039 |
| SER | 1.2 | 0.304 | 1.5 | 0.270 | 0.1 | 0.707 | 1.1 | 0.386 | 1.4 | 0.255 |
| HIS | 1.1 | 0.313 | 0.6 | 0.675 | 0.4 | 0.542 | 2.9 | 0.019 | 1.7 | 0.155 |
| ADO | 0.0 | 0.952 | 1.1 | 0.401 | 0.2 | 0.700 | 0.7 | 0.645 | 2.2 | 0.067 |
| CORT | 1.1 | 0.333 | 4.3 | 0.008 | 1.5 | 0.243 | 1.3 | 0.286 | 1.9 | 0.101 |

Phenylethylamine, PEA; Phenylalanine, PHE; tyrosine, TYR; 3,4-dihydroxyphenylalanine, L-DOPA; dopamine, DA; 3-methoxytyramine, 3-MT; 3,4-dihydroxyphenylacetic acid, DOPAC; homovanillic acid, HVA; norepinephrine, NE; 3,4-dihydroxyphenylglycol, DHPG; 3-methoxy-4-hydroxyphenylglycol, MHPG; normetanephrine, NM; metanephrine, MN; vanillylmandelic acid, VMA; Tryptophan, TRP, kynurenine, KYN; kynurenic acid, KA; xanthurenic acid, XA; quinolinic acid, QA; 5-hydroxytryptophan, 5-HTP; serotonin, 5-HT; 5-hydroxyindoleacetic acid, 5-HIAA; melatonin, MT; Glutamic acid, GLU; alpha-aminobutyric acid, AABA; and gamma-aminobutyric acid, GABA. Other endogenous analytes associated with the gut brain axis (GBA) measured were glycine, GLN; serine, SER, histamine, HIS; adenosine, ADO; cortisol, CORT.

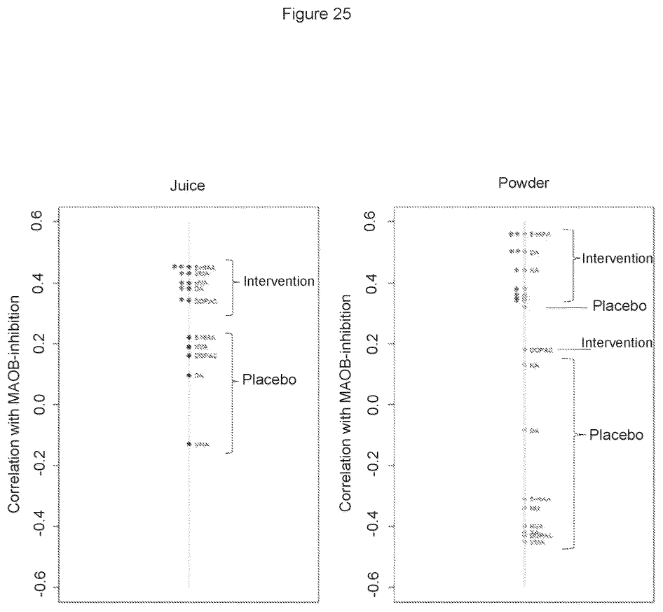

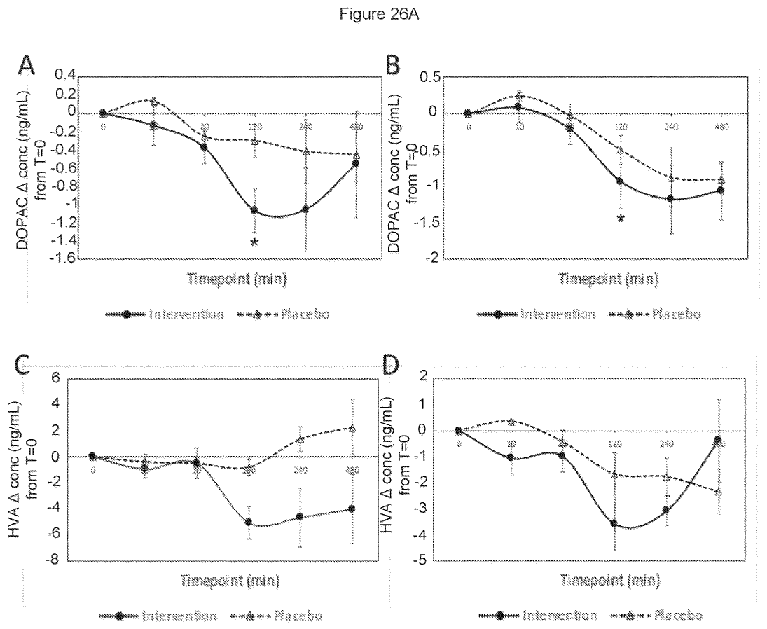

To identify if any modulation of plasma neurotransmitter concentrations might be correlated with MAO-B inhibition, pairwise rank-sum correlation testing was performed. Four neurotransmitters from the tyrosine metabolic pathway, DOPAC, VMA, HVA and DA, and one from the tryptophan metabolic pathway, 5-HIAA showed significant positive correlations with MAO-B inhibition following consumption of the blackcurrant juice format (FIG. 25). The same correlations for the placebo intervention were not significant. Except for DA, these neurotransmitters are MAO-derived metabolites, and suggests the downstream effect of BC-induced MAO-B inhibition on circulating neurotransmitter concentrations.

Aside from DOPAC, these same neurotransmitters also showed significant positive correlations with MAO-B inhibition following consumption of the blackcurrant powder, that were not observed for the placebo. For the powder intervention format only, significant correlations with MAO-B inhibition were also observed for XA, MN, NM and KA, metabolites from the tryptophan metabolic pathway. While NM is a substrate for MAO-A/B, XA, MN and KA are not substrates or metabolic products of MAO-A/B.

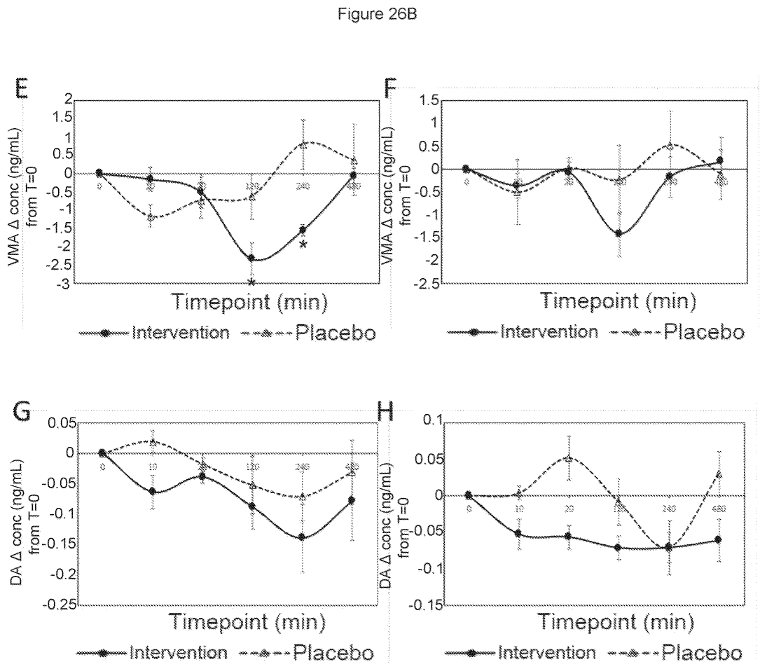

The temporal changes in plasma neurotransmitters concentrations from the tyrosine metabolomic pathway following BC-JC and BC-P interventions and their placebos are plotted on FIG. 26. Plasma DOPAC was significantly lower 120 min post BC-JC and BC-P consumption compared with the placebo group at the same timepoint (FIG. 26A-B). Plasma VMA concentrations were significantly lower 120 min and 240 min after BC-JC, but not BC-P, consumption compared with the placebo group at these timepoints (FIG. 26E-F). Although plasma HVA and DA concentrations tended to be lower following BC-JC and BC-P consumption compared with their corresponding placebos, no significant treatment differences were measured at any of the timepoints for these neurotransmitters.

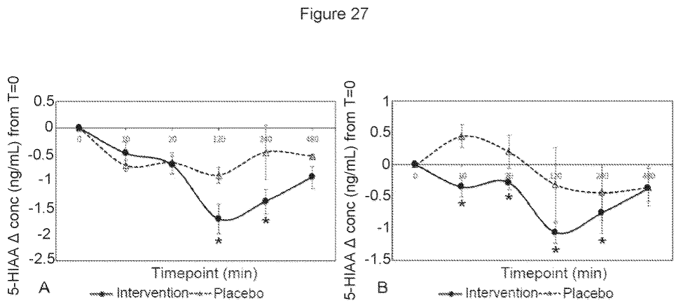

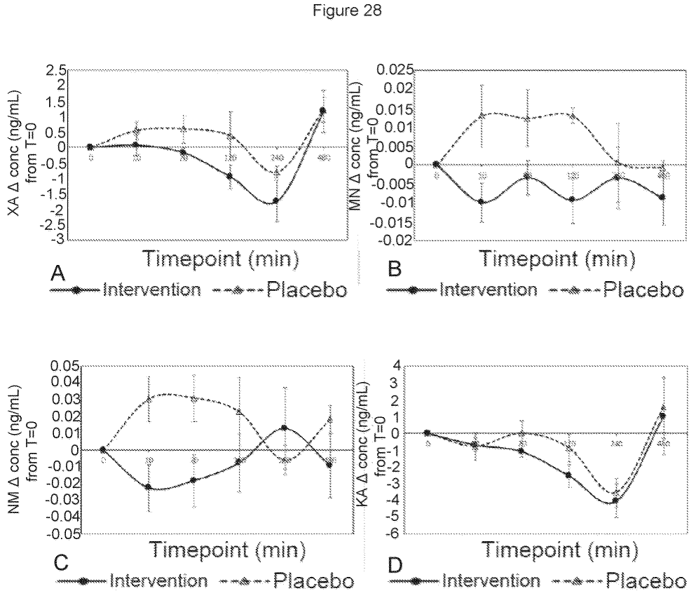

Blackcurrant consumption was also found to temporally modulate plasma concentrations of 5-HIAA, a tyrosine metabolite (FIG. 27). Significantly lower plasma 5-HIAA concentrations were measured as early as 10 min after BC-P consumption which persisted up to 240 min compared with the placebo group. This near-immediate effect was not measured in the BC-JC group, where significant reduction in plasma 5-HIAA was observed at 120 and 240 min compared with the placebo group. Correlation analysis also revealed significant correlations between XA, MN, NM and KA with MAO-B for BC-P only. Further ANOVA analysis revealed no significant time and treatment interactions of these neurotransmitters following BC-P consumption at any of the measured timepoints (FIG. 28).

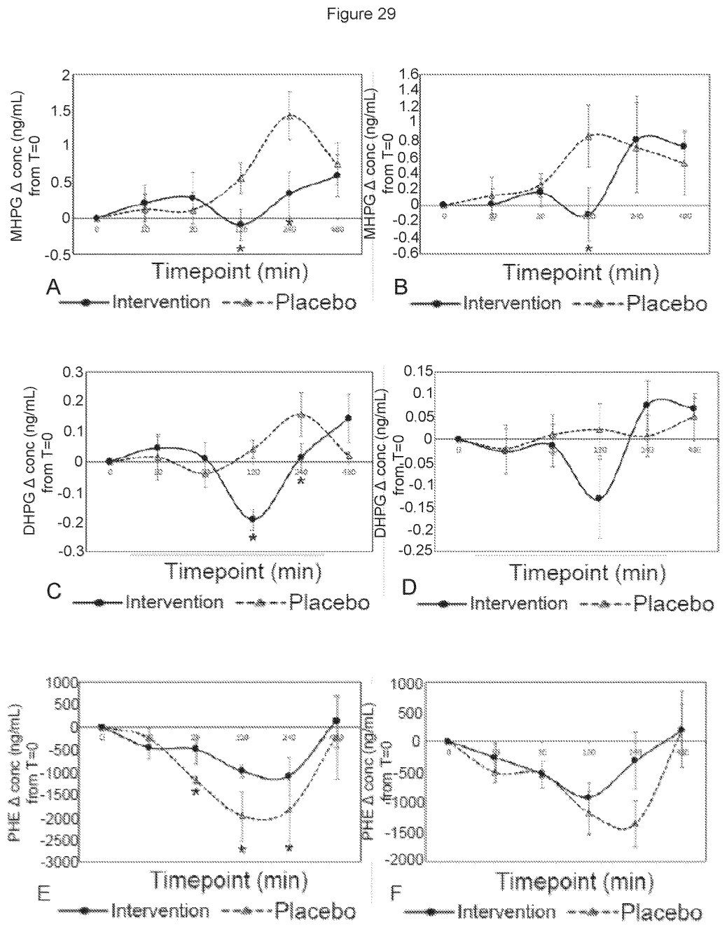

Although not significantly correlated to MAO-B inhibition, univariate ANOVA analysis revealed a significant time×treatment interaction for the neurotransmitters MHPG, DHPG and PHE (Table 17). The relative change in the concentration of these neurotransmitters following BC-JC and BC-P consumption is plotted in FIG. 29 to illustrate the variation in the effect of format on these neurotransmitters. Significantly lower plasma MHPG concentrations was measured 120 min post BC-JC and BC-P consumption compared with the placebo group. However, consuming a single dose of BC-JC, but not BC-P, significantly reduced plasma DHPG (120-240 min) and PHE (20-240 min) compared with the placebo group at these timepoints.

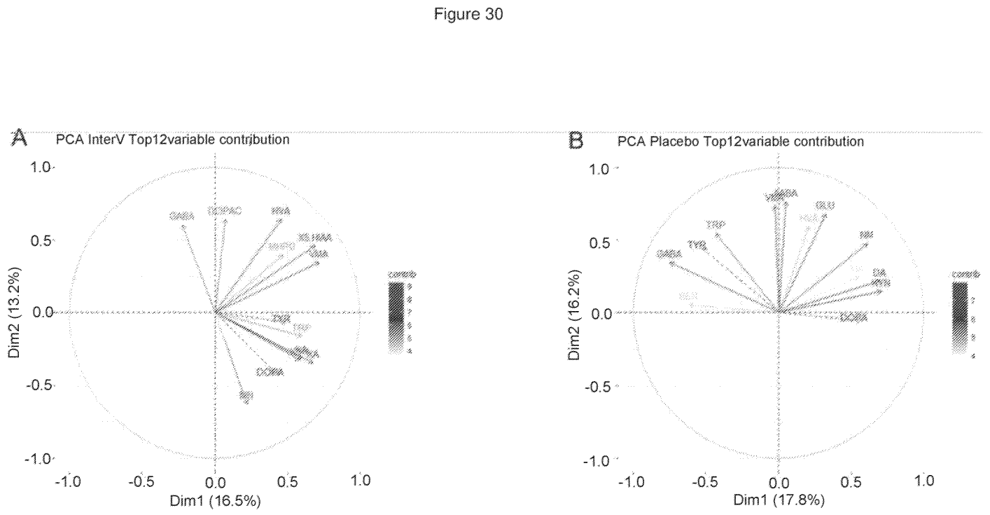

To further explore which neurotransmitter combinations might drive the variation between timepoints across both blackcurrant formats compared with placebo, a multivariate approach was employed. The correlation circle plots (FIG. 30) display the correlations between the top 12 variables with the strongest contributions to the principal components. FIG. 30A shows a primary contribution of 5-HIAA to drive variation in neurotransmitter concentrations during the time-course experiments associated with the blackcurrant intervention (juice and powder combined). It further suggests a strong interaction between 5-HIAA, HVA, VMA, and association with DOPAC and MHPG. These associations were not observed for the placebo intervention (FIG. 30B)

### Discussion

This study demonstrated that despite the difference in anthocyanin dose consumed by participants of the two different BC format groups (juice concentrate and powder) consuming a single 300-mL beverage prepared from either format resulted in equivalent inhibition of platelet MAO-B enzyme activity. Blackcurrant anthocyanins were detectable in plasma 10 min post-consumption of both formats, peaked at 120 min, then progressively declined thereafter to near-baseline concentrations 480 min post-consumption. Higher concentrations of individual and total anthocyanins were measured in plasma following consumption of the BC powder beverage than with the BC juice concentrate beverage. In contrast, no differences in the bioavailability of sarmentosin was measured between the two blackcurrant formats at any timepoints. Significant changes in plasma neurotransmitter concentrations were measured following consumption of the BC juice concentrate and BC powder beverages compared to placebo. The findings also suggest the efficacy of consuming the blackcurrant in regulating participants' mood or other beneficial cognitive factors, at least partially through MAO enzyme inhibition through sarmentosin activity.

The concentrations of cyanidin and delphinidin derivatives measured in the plasma of BC-P were approximately seven times higher than the concentrations for BC-JC. This difference in bioavailability is attributed to the different doses of anthocyanins consumed by the participants: on average, the anthocyanin dose consumed by participants in the BC-P group was almost eight times the amount consumed by those in the BC-JC group. Despite the differences in dose, the temporal bioavailability profiles of total and individual anthocyanins during the trial day were similar for both BC-P and BC-JC, suggesting that the rate of absorption of anthocyanins is similar for both formats.

Sarmentosin bioavailability was also measured in plasma samples using standards provided by Stephen Bloor from Callaghan Innovation. The bioavailability of sarmentosin in plasma after consuming the two blackcurrant formats was similar and followed the same bioavailability profile as that of anthocyanins. Interestingly, the bioavailability of sarmentosin at each of the timepoints measured was similar between formats, despite the large difference in anthocyanin dose consumed. It should be noted that the recovery of sarmentosin in all plasma samples was low and the sarmentosin results presented in this report were not corrected for recovery. Further work to refine the methodology and increase sarmentosin recovery in plasma is required to more accurately quantify sarmentosin in plasma.

This study demonstrated the rapid, sustained and significant reduction in peripheral platelet MAO-B activity after consuming a single dose of the tested blackcurrant beverages (BC-JC & BC-P). The inhibition of MAO-B activity in platelets also aligned with the bioavailability profile of anthocyanins in plasma. MAO-B activity correlated with the increasing bioavailability of anthocyanins in plasma, while the decline in bioavailable anthocyanins following 120 min after BC consumption was concomitant with reduced MAO-B inhibition.

While the MAO-B inhibitory bioactivity of anthocyanins cannot be dismissed, it is plausible that significant platelet MAO-B inhibition observed in these clinical studies may be largely attributed to blackcurrant constituents other than anthocyanins, for example sarmentosin.

Analysis of the subjective data indicate a potential effect of the BC beverages in modulating mood in parallel with increased sarmentosin concentration plasma levels, and also MAO enzyme inhibition. Consumption of blackcurrant juice and powder variably reduced stress and anxiety of participants, reduced participant calmness compared to placebo, and either maintained or significantly reduced subjective scores of mental fatigue. Greater reductions of stress, anxiety, and mental fatigue were measured in the BC-P group compared with the BC-JC group. A general increase in alertness was measured immediately after consuming all beverages, which then progressively decreased over the course of the trial day.

Alertness measured after consuming BC-JC remained consistent over the course of the trial day, whereas alertness after PL-JC dropped to below baseline values at 480 min. And alertness after BC-P consumption remained elevated and was significantly higher than after PL-P at 120 min. Taken together, these findings suggest that blackcurrant consumption improved alertness, or prevented its decline, over the course of the trial day.

In most mammalian tissue, including humans, MAOs are present as two isoforms, MAO-A and MAO-B, which display regional differences in enzyme activity, substrate specificity and distribution in the brain and periphery (Yeung et al. 2019). 5-HT is reported to be preferably degraded by MAO-A, whereas MAO-B exhibits higher affinity towards benzylamine and PEA. Catecholamines such as DA, E, NE, tryptamine and 3-MT are substrates for both isoforms (Goldstein et al. 2021). Further, plasma concentrations of DHPG and MHPG have been described as sensitive indicators of MAO-A dependent metabolism of NE (Scheinin et al. 1991).

Initial multivariate analysis identified five neurotransmitters that showed significant correlations with MAO-B inhibition with both juice and powder format interventions, and a further four that showed a significant correlation for the powder format only. Three (5-HIAA, DOPAC and VMA) showed consistent treatment×time effects that were statistically significant in the juice format only. Further analysis identified a strong interaction between 5-HIAA, HVA, VMA and association with DOPAC and MHPG. Our findings also confirmed a significant treatment×time effect for MHPG, and its precursor DHPG, although the latter only reached a level of significance for the juice format. The treatment×time effect for HVA (p=0.024) was not statistically significant, but the change in concentration profile was very similar to observations for VMA, particularly for the juice intervention format. Significant changes in concentration for each of these analytes were maximal at 120 min post-ingestion of the treatment, with lower plasma concentrations in the blackcurrant groups relative to the placebo. Of note, the neurotransmitters highlighted by our analysis are all end-products of MAO-metabolized neurotransmitters as part of the tyrosine and tryptophan metabolic pathways.

Interestingly, declines in the circulating concentrations of 5-HIAA and MHPG/DHPG following blackcurrant consumption suggests the benefits of blackcurrants inhibiting both MAO-A and MAO-B activity.

Despite both blackcurrant juice and powder interventions inhibiting platelet MAO-B activity to a similar degree, differences in the modulation of some neurotransmitters were observed between the two formats. The neurotransmitters VMA, DHPG and PHE were significantly reduced following BC-JC, but not BC-P, consumption even when a higher anthocyanin dose was consumed by those in the BC-P intervention. It is possible that these differences may be minimised by testing the efficacy of these formats in a larger sample size. It is also plausible that these format differences may be attributed to variation of bioactives, other than anthocyanins, between the juice and powder formats.

The small sample size of participants enrolled to each BC format is an important limitation to be considered when interpreting mood and neurotransmitter data. The number of participants recruited for this study was based on detecting differences in MAO-B inhibition as a primary outcome, and mood and neurotransmitter data as secondary outcomes. To account for this limitation, a stringent approach was taken in the final analysis of our neurotransmitter results to determine whether any treatment effects or interactions were statistically significant (p<0.01). Another limitation is the environmental influences that were not controlled that may have influenced participants' moods during the trial days, as they were allowed to choose to come and go from the facility following the 20-min data collection timepoint.

The significant differences in blood glucose concentrations measured between BC-JC and PL-JC might be due to differences in the sugar content between the two interventions. To ensure that the sugar contents between the two doses were equivalent, we formulated the BC-JC and PL-JC to the same soluble solids content (measured as °Brix). However, it should be noted that Brix is only an approximate measurement for sugar content and may be influenced by other solids that are present in the solution. Anthocyanins are also known to modulate blood glucose concentrations (Kim et al. 2016), which may partly explain the reduction in blood glucose concentrations observed in the anthocyanin-rich BC-JC intervention compared with the PL-JC intervention.

### CONCLUSIONS AND FUTURE WORK

The results showed very similar bioavailability profiles between the blackcurrant juice concentrate and powder interventions, although at different magnitudes. The temporal bioavailability of sarmentosin, a specific metabolite of interest for this study, also followed a very similar bioavailability profile between the two blackcurrant formats up to 480 min after consumption.

The results demonstrated that consumption of beverages prepared from blackcurrant powder and juice concentrate reduced platelet MAO-B enzyme activity by the same magnitude and at the same rate, despite the different formats and dose approaches used for each format. To the Applicant's knowledge, this is the first study to demonstrate the efficacy of a blackcurrant powder in inhibiting platelet MAO-B to the same magnitude as blackcurrant juice. The MAO-B activity data in conjunction with the bioavailability data indicate that a lower dose of blackcurrant anthocyanins in a beverage formulated from a juice concentrate causes an equivalent MAO-B inhibition to that of a higher dose from a blackcurrant powder-formulated beverage.

The inhibition of platelet MAO-B activity following consuming a single dose of BC juice or powder was also concomitant with significant transient reduction of circulating monoamine neurotransmitters DOPAC, 5-HIAA and MHPG. We also observed format effects on monoamine neurotransmitters with statistically significant reductions in circulating VMA and DHPG following BC juice, but not BC powder, consumption. The reasons underpinning these format effects are unclear but may be due to the small sample size of this study or compositional differences between the two formats.

Subjective data collected during this study suggest the benefit of consuming blackcurrant for reducing resting stress, anxiety and mental fatigue, as well as improving alertness.

### REFERENCES

Watson A W, Haskell-Ramsay C F, Kennedy D O, Cooney J M, Trower T, Scheepens A. Acute supplementation with blackcurrant extracts modulates cognitive functioning and inhibits monoamine oxidase-B in healthy young adults. J Funct Foods 2015; 17:524-39.
Watson A W, Scheepens A, Kennedy D O, Cooney J M, Trower™, Haskell-Ramsay C F. The pharmacodynamic profile of “Blackadder” blackcurrant juice effects upon the monoamine axis in humans: A randomised controlled trial. Nutr Neurosci. 2020 July; 23(7):516-525. doi: 10.1080/1028415X.2018.1525950. Epub 2018 Oct. 5. PMID: 30289026.
Herraiz, T., Chaparro, C. (2006) Analysis of monoamine oxidase enzymatic activity by reversed-phase high performance liquid chromatography and inhibition by β-carboline alkaloids occurring in foods and plants. Journal of Chromatography A 1120(1-2), pp. 237-243
Herraiz, T., Flores, A., Fernández, L. (2018). Analysis of monoamine oxidase (MAO) enzymatic activity by high-performance liquid chromatography-diode array detection combined with an assay of oxidation with a peroxidase and its application to MAO inhibitors from foods and plants. Journal of Chromatography B: Analytical Technologies in the Biomedical and Life Sciences 1073, pp. 136-144
Blech, S., Budzikiewicz, H., Dill, D. (1994). B-Carboline Alkaloids from Ribes nigrum L. Zeitschrift fur Naturforschung-Section C Journal of Biosciences. 49(9-10), pp. 540-544
Yamada M, Yasuhara H. Clinical pharmacology of MAO inhibitors: safety and future. Neurotoxicology. 2004 January; 25(1-2):215-21. doi: 10.1016/S0161-813X(03)00097-4. PMID: 14697896.
Delumeau J. C., Bentué-Ferrer D., Gandon J. M., Amrein R., Belliard S., Allain H. (1994) Monoamine oxidase inhibitors, cognitive functions and neurodegenerative diseases. In: Tipton K. F., Youdim M. B. H., Barwell C. J., Callingham B. A., Lyles G. A. (eds) Amine Oxidases: Function and Dysfunction. Journal of Neural Transmission, vol 41. Springer, Vienna.
Cai, Z. “Monoamine oxidase inhibitors: Promising therapeutic agents for Alzheimer's disease (Review)”. Molecular Medicine Reports 9, no. 5 (2014): 1533-1541.
Carradori S, Secci D, Petzer J P. MAO inhibitors and their wider applications: a patent review. Expert Opin Ther Pat. 2018 March; 28(3):211-226. doi: 10.1080/13543776.2018.1427735. Epub 2018 Jan. 17. PMID: 29324067.
Mallinger A G, Smith E. Pharmacokinetics of monoamine oxidase inhibitors. Psychopharmacol Bull. 1991; 27(4):493-502. PMID: 1813896.
Herraiz T. Identification and occurrence of beta-carboline alkaloids in raisins and inhibition of monoamine oxidase (MAO). J Agric Food Chem. 2007 Oct. 17; 55(21):8534-40. doi: 10.1021/jf0719151. Epub 2007 Sep. 21. PMID: 17883257.
Dingemanse J, Wood N, Jorga K, Kettler R. Pharmacokinetics and pharmacodynamics of single and multiple doses of the MAO-B inhibitor lazabemide in healthy subjects. Br J Clin Pharmacol. 1997 January; 43(1):41-7. PMID: 9056051.
Lu Y, Foo L Y, Wong H. Nigrumin-5-p-coumarate and nigrumin-5-ferulate, two unusual nitrile-containing metabolites from black currant (Ribes nigrum) seed. Phytochemistry. 2002 February; 59(4):465-8. doi: 10.1016/s0031-9422(01)00441-1. PMID: 11830168.
Fernandes J M, Termentzi A, Mandova T, Hammad K, Machera K, Magiatis P, Michel S, Zucolotto S M, Grougnet R. Detection, Isolation, and 1H NMR Quantitation of the Nitrile Glycoside Sarmentosin from a Bryophyllum pinnatum Hydro-Ethanolic Extract. J Agric Food Chem. 2021 Jul. 28; 69(29):8081-8089. doi: 10.1021/acs.jafc.1c01414. Epub 2021 Jul. 19. PMID: 34279926.
Anima Ghosal, Chapter 8-Evaluation of the clearance mechanism of non-CYP-mediated drug metabolism and DDI as a victim drug, Editor(s): Shuguang Ma, Swapan K. Chowdhury, Identification and Quantification of Drugs, Metabolites, Drug Metabolizing Enzymes, and Transporters (Second Edition), Elsevier, 2020, Pages 237-271, ISBN 9780128200186, https://doi.org/10.1016/B978-0-12-820018-6.00008-9.
Bond A, Lader M 1974. The use of analogue scales in rating subjective feelings. British Journal of Medical Psychology 47(3): 211-218.
Goldstein D S, Castillo G, Sullivan P, Sharabi Y 2021. Differential Susceptibilities of Catecholamines to Metabolism by Monoamine Oxidases. J Pharmacol Exp Ther 379(3): 253-259.
Kim Y, Keogh J B, Clifton P M 2016. Polyphenols and Glycemic Control. Nutrients 8(1).
Lomiwes D, Ha B, Ngametua N, Burr N S, Cooney J M, Trower T M, Sawyer G, Hedderley D, Hurst R D, Hurst S M 2019. Timed consumption of a New Zealand blackcurrant juice support positive affective responses during a self-motivated moderate walking exercise in healthy sedentary adults. J Int Soc Sport Nutr 16(1).
Parkar S G, Jobsis CMH, Trower™, Cooney J M, Hedderley D I, Bentley-Hewitt K L 2020. Bioaminergic Responses in an In Vitro System Studying Human Gut Microbiota-Kiwifruit Interactions. Microorganisms 8(10).
Scheinin M, Karhuvaara S, Ojalakarlsson P, Kallio A, Koulu M 1991. Plasma 3,4-Dihydroxyphenylglycol (Dhpg) and 3-Methoxy-4-Hydroxyphenylglycol (Mhpg) Are Insensitive Indicators of Alpha-2-Adrenoceptor Mediated Regulation of Norepinephrine Release in Healthy-Human Volunteers. Life Sci 49(1): 75-84.
Watson A W, Okello E J, Brooker H J, Lester S, McDougall G J, Wesnes K A 2019. The impact of blackcurrant juice on attention, mood and brain wave spectral activity in young healthy volunteers. Nutr Neurosci 22(8): 596-606.
Yeung A W K, Georgieva M G, Atanasov A G, Tzvetkov N T 2019. Monoamine Oxidases (MAOs) as Privileged Molecular Targets in Neuroscience: Research Literature Analysis. Front Mol Neurosci 12.

1-7. (canceled)

8. A composition formulated as a food or beverage, pharmaceutical composition, nutraceutical or an extract from a plant material, wherein the composition includes sarmentosin or ester(s) thereof, in an effective amount for inhibiting monoamine oxidase enzyme A (MAO-A) and/or monoamine oxidase enzyme B (MAO-B).

9. The composition as claimed in claim 8 wherein the effective amount of sarmentosin is at least 0.005 mg/g in the composition.

10. The composition as claimed in claim 8 wherein the effective amount of sarmentosin is between about 0.005 to 70 mg/g in the composition.

11-13. (canceled)

14. A method of inhibiting monoamine oxidase enzyme A (MAO-A) and/or monoamine oxidase enzyme B (MAO-B) comprising administering to a person in need thereof an effective amount of sarmentosin or ester(s) thereof, or a plant material, an extract or a composition containing same.

15. The method as claimed in claim 14, wherein the administering treats or prevents disease or condition of the person associated with MAO-A or MAO-B enzyme activity.

16. The method as claimed in claim 14, wherein the administering improves, maintains or prevents a non-clinical outcome of the person selected from the group consisting of mood, anxiety, social anxiety, fatigue, cognitive performance or function, exercise performance, attention/alertness, calmness, mental clarity, executive function, working memory, secondary memory, mood, stress, stress reactivity, nootropic effects and combinations thereof.

17. The the method as claimed in claim 14, wherein the method modulates, maintains or increases a neurotransmitter selected from the group consisting of dopamine, serotonin, adrenalin and tyramine.

18. The method of claim 15 wherein the disease or condition is any neurological or psychiatric condition or ailment associated with MAO-A or MAO-B enzyme activity.

19. The method of claim 15, wherein the disease or condition is selected from the group consisting of depression, atypical depression, panic, social anxiety, bipolar disorder, post-traumatic stress disorder (PTSD), Obsessive Compulsive Disorder (OCD), adult ADHD, Alzheimer's disease, Dementia, Parkinson's disease and Huntington's disease.

20. The method as claimed in claim 14, wherein the person has a non-clinical condition and the daily dosage of sarmentosin or its ester(s) thereof is at least 1 mg, and more preferably between 1 to 20 mg.

21. The method as claimed in claim 14, wherein the person has a clinical disease or condition associated with MAO-A or MAO-B enzyme activity and the daily dosage of sarmentosin or its ester(s) thereof is at least 20 mg.

22. The method as claimed in claim 14, wherein the person has a clinical disease or condition associated with MAO-A or MAO-B enzyme activity and the daily dosage of sarmentosin or its ester(s) thereof is between 20 to 200 mg for a clinical use, or more preferably between 20 to 80 mg.

23. The composition of claim 8, which is a blackcurrant fruit, blackcurrant juice, blackcurrant extract, or isolate including an effective amount of sarmentosin or ester(s) thereof for inhibiting MAO-A or MAO-B.

24. The method as claimed in claim 14, wherein the administering is performed for at least for one day for a non-clinical use, or at least once per day for at least six weeks for a clinical use.

25. A method of making a composition for inhibiting a disease or condition associated with MAO-A or MAO-B comprising synthesizing or extracting sarmentosin or an ester(s) thereof from a plant source and incorporating the sarmentosin or ester(s) thereof into a composition in an effective amount for treating the disease or condition.
